(19) United States
(12) Patent Application Publication
(10) Pub. No.: US 2022/0105157 A1
(43) Pub. Date: Apr. 7, 2022

(54) Compositions and Methods for Promoting Angiogenesis in the Eye

(71) Applicant: The Regents of the University of California, Oakland, CA (US)

(72) Inventors: Napoleone Ferrara, La Jolla, CA (US); Qin Li, La Jolla, CA (US); Pin Li, La Jolla, CA (US)

(21) Appl. No.: 17/419,151

(22) PCT Filed: Dec. 26, 2019
(86) PCT No.: PCT/US2019/068595

§ 371 Date: Jun. 28, 2021

Related U.S. Application Data
(60) Provisional application No. 62/788,174, filed on Jan. 4, 2019.

(51) Int. Cl. A61K 38/20; A61P 9/10; A61P 9/12; A61P 27/02

(52) U.S. Cl. CPC A61K 38/2093; A61P 9/10; A61K 9/0048; A61P 27/02; A61K 38/204; A61P 9/12

(57) ABSTRACT

Compositions and methods for promoting angiogenesis in the eye with IL-6 family proteins, including leukemia inhibitory factor (LIF) or cardiotrophin-1 (CT-1) are provided.

### CROSS REFERENCES TO RELATED APPLICATIONS

This application claims the priority benefit of U.S. Provisional Application No. 62/788,174, filed Jan. 4, 2019, which is incorporated herein by reference.

### TECHNICAL FIELD

The present invention relates to promotion of angiogenesis to alleviate conditions of the eye.

### BACKGROUND

Angiogenesis is a physiological process required for embryonic development, adult vascular homeostasis, and tissue repair (1). Yet, angiogenesis also contributes to a variety of pathological conditions such as tumors and several intraocular disorders including wet age-related macular degeneration (AMD) (1). During tumor progression, the new vessels provide neoplastic tissues with nutrients and oxygen and thus play an essential role; in intraocular disorders, growth of abnormal, leaky blood vessels may destroy the retina and lead to blindness (1, 2). Extensive efforts to dissect the molecular basis of angiogenesis and to identify therapeutic targets for neoplasms and other diseases resulted in the discovery of key signaling pathways involved in vascular development and differentiation (1, 3). In particular, numerous studies have established the pivotal role of the VEGF pathway in physiological angiogenesis and therapies targeting this pathway have achieved success in treatments of cancer and ocular disorders such as wet AMD (4, 5). Conversely, stimulating angiogenesis holds the promise of improving outcomes of patients with a variety of ischemic disorders through improved perfusion (6). This hypothesis led to a series of clinical trials in the past decades, testing angiogenic factors such as VEGF or bFGF, delivered by gene therapy or as recombinant proteins in coronary or limb ischemia patients. Unfortunately, none of these studies were successful, in spite of promising preclinical studies (7). Therefore, there is a need to identify novel strategies to improve angiogenic therapy.

Glioblastoma cells secrete a variety of angiogenic factors, which contribute to the highly vascular phenotype of such tumors (8). Xenograft tumors derived from the LN-229 glioblastoma cell line are adequately vascularized in spite of a very low VEGF expression (9, 10). Therefore, the LN-229 secretome is of interest to characterize putative endothelial mitogens.

The IL-6 superfamily of cytokines includes Leukemia Inhibitory Factor (LIF). It is widely used in experimental stem cell biology due to its ability to maintain the pluripotency of embryonic stem cells. A variety of roles of LIF in different types of cells and tissues have also been observed, including embryo implantation, hematopoietic cell development, inflammatory responses, tumor progression, etc. (67).

The role of LIF in angiogenesis is still matter of debate. It was initially characterized as an anti-angiogenic factor on bovine aortic endothelial cells and showed no effect on bovine adrenal cortex capillary endothelial cells (35), suggesting that LIF functions distinctively on different types of endothelial cells. Subsequent studies showed a considerable complexity. Transgenic mice overexpressing LIF showed reduced vasculature in the eye and suppressed retinal vascular development (14); while mice carrying homozygous LIF knockout alleles had increased vessel density in the retina (16). Injection of recombinant LIF into rat pups at the early postnatal period also resulted in slightly increased avascular area in developing retina (22).

### SUMMARY OF THE INVENTION

The present invention provides that members of the IL-6 superfamily, and functional fragments thereof, can be used to increase angiogenesis in the eye of a subject in need to therapeutically treat conditions such as, but not limited to age-related macular degeneration and retinopathy of prematurity (ROP). In embodiments, the subject is a human.

In embodiments, the invention provides a method of treatment for a condition related to inadequate vascularization in the eye of a subject comprising administering to a subject in need thereof an effective amount of an IL-6 family protein, or a functional fragment thereof, to promote angiogenesis. In embodiments, the invention provides that the IL-6 family protein is leukemia inhibitory factor (LIF) or cardiotrophin-1 (CT-1).

In embodiments, the invention provides that the administration increases retinal microvessel density. In embodiments, the invention provides that the administration increases proliferation of choroidal endothelial cells.

In embodiments, the invention provides that the condition is age-related macular degeneration. In embodiments, the invention provides that the condition is retinopathy of prematurity (ROP).

In embodiments, the invention provides that the administration is via intravitreal injection. In embodiments, the invention provides that the effective amount does not induce vascular leakage. In embodiments, the invention provides that the effective amount does not induce edema.

In embodiments, the invention provides a method of inducing blood vessel formation in the eye of a subject comprising administering to a subject in need thereof an effective amount of an IL-6 family protein, or a functional fragment thereof.

In embodiments, the invention provides that the administration increases retinal angiogenesis. In embodiments, the invention provides that the administration increases proliferation of choroidal endothelial cells.

In embodiments, the invention provides that the subject has age-related macular degeneration. In embodiments, the invention provides that the subject has retinopathy of prematurity (ROP).

In embodiments, the invention provides that the administration is via intravitreal injection. In embodiments, the invention provides that the effective amount does not induce vascular leakage. In embodiments, the invention provides that the effective amount does not induce edema.

In embodiments, the invention provides that the IL-6 family protein is leukemia inhibitory factor (LIF). In embodiments, the invention provides that the IL-6 family protein is cardiotrophin-1 (CT-1).

### BRIEF DESCRIPTION OF THE DRAWINGS

FIGS. 1A-1F show LIF is the endothelial cell mitogen from LN-229 conditioned medium. LN-229 conditioned medium stimulates growth of bovine choroidal endothelial cells, n=3 (FIG. 1A). VEGF neutralizing antibody fails to suppress BCE cell growth induced by LN-229 CM, n=3 (FIG. 1B). Reverse-phase chromatography fractions of LN-229 CM induce BCE cell growth. BCE cells were incubated with fractions (2 μl/well) as indicated in the figure, n=3 (FIG. 1C). The anti-LIF neutralizing antibody abolishes BCE cell growth induced by reverse-phase fractions, n=3 (FIG. 1D). Recombinant human LIF proteins stimulate growth of BCE cells in a dose-dependent manner. BCE cells were cultured in the presence of vehicle, VEGF (10 ng/ml) and the indicated concentrations of recombinant human LIF (rhLIF), n=3 (FIG. 1E). LIF and VEGF synergistically stimulate BCE cell growth. Cell proliferation was analyzed after 6 days using alamar blue, n=3. Bars and error bars represent mean±SD. *, p<0.05; **, p<0.01; #, p≤0.0001; ns, not statistically significant (FIG. 1F).

FIGS. 2A-2E show LIF promotes BCE cell growth via the JAK-STAT3 pathway. The JAK inhibitor baricitinib (Ba) blocks activation of STAT3 by LIF. BCE cells were pre-incubated with DMSO, baricitinib (2 μM), cobimetinib (Co) (150 nM) or BEZ235 (BE) (5 nM) for 1 hour and then treated with vehicle or LIF (10 ng/ml) for 15 minutes. Ctrl, no pre-incubation with inhibitors (FIG. 2A). Baricitinib suppresses LIF-induced BCE cell growth. BCE cells were pre-incubated with DMSO, baricitinib, cobimetinib, or BEZ235 for 1 hour and then treated with vehicle, LIF (10 ng/ml) or VEGF (10 ng/ml). Cell proliferation was analyzed after 6 days, n=3 (FIG. 2B). FIGS. 2C and 2D show STAT3 knockdown in BCE cells. BCE cells were transfected with siNegative and siRNAs targeting STAT3. qRT-PCR were performed to examine STAT3 mRNA levels. STAT3 level in siNegative was set as 1. Data from three independent experiments were averaged and are presented in FIG. 2C. In FIG. 2D, cells transfected with siRNAs were treated with LIF (10 ng/ml) or vehicle for 15 minutes. Whole-cell lysates were subjected to Western blotting with the indicated antibodies. LIF-induced BCE cell growth was abolished by STAT3 knockdown. BCE cells with STAT3 knockdown were cultured with LIF (10 ng/ml) or vehicle. Cell proliferation was analyzed after 3 days. Fluorescence reading at 590 nm for each vehicle group was set as 1, n=3. siNegative, negative control siRNA not targeting any known genes. **, p<0.01; ***p<0.001; #, p≤0.0001; ns, not statistically significant (FIG. 2E).

FIGS. 3A-3J show that LIF promotes angiogenesis in ex vivo and in vivo models. FIGS. 3A and 3B show induction of mouse choroidal sprouting by LIF. Vascular proliferation from primary choroidal explants at 6 days post-seeding are shown in the representative pictures of FIG. 3A. Supplements were added to each sample as indicated. Quantification of the growth of vascular sprouts was performed using Axiovision software, n=5. FIGS. 3C and 3D show that intravitreal injection of LIF increases vessel density in mouse eyes. Adult mice were intravitreally injected indicated amounts of VEGF and LIF. Seven days after injection, PFA-fixed choroid-sclera complexes and retina were subjected to CD31 IF. Representative images of CD31-positive vessels are shown in FIG. 3C. Vascular density determined with ImageJ software is presented in FIG. 3D, n=5-8. FIGS. 3E and 3F show OCTA imaging of LIF-treated mouse retina. Adult mice were intravitreally injected with 1 μl of LIF (50 ng) or vehicle solution (PBS). Retinal OCTA images were obtained 7 days after the injection and representatives are shown in FIG. 3E. Vessel density was determined with percentage of vessel-covered area/total area surface using ImageJ software and shown in FIG. 3F, n=7-8. FIGS. 3G and 3H show that LIF treatment increases vessel density in mouse retina. Adult mice were intravitreally injected with LIF (10 ng) or vehicle solution. Seven days after injection, frozen sections of mouse eyes were subjected to H&E staining and CD31 IF staining. Representative images are shown in FIG. 3G. Quantification of CD31-positive using ImageJ software is shown in FIG. 3H, n=4. In FIGS. 3I and 3J, five-day old neonatal mice were intravitreally injected with LIF (50 ng) or vehicle solution (PBS). Upon treatment for 3 days, mouse retinas were subjected to IF staining with Dyight-488-labeled lectin. Representative images for similar ocular loci are shown in FIG. 3I. Quantification of lectin-labeled area using ImageJ software is shown in FIG. 3J, n=4. *, p<0.05; **, p<0.01.

FIGS. 4A-4F show that LIF inhibits BAE cell growth through the JAK-STAT3 pathway. Recombinant human LIF inhibits growth of BAE cells in a dose-dependent manner. BAE cells were cultured in the presence of vehicle and indicated concentrations of recombinant human LIF (rhLIF). Cell proliferation was analyzed after 6 days, n=3 (FIG. 4A). JAK inhibitor baricitinib blocks activation of STAT3 by LIF. BAE cells pre-incubated with DMSO and inhibitors for 1 hour were treated with vehicle and LIF (10 ng/ml) for 15 minutes. Whole-cell lysates were subjected to Western blotting with indicated antibodies. Ctrl, no pre-incubation with inhibitors; Ba, baricitinib (2 μM); Co, cobimetinib (150 nM); BE, BEZ235 (5 nM) (FIG. 4B). The JAK inhibitor baricitinib reverses LIF-induced BAE growth inhibition. BAE cells pre-incubated with inhibitors for 1 hour were treated with vehicle, LIF (10 ng/ml) and VEGF (10 ng/ml). Cell proliferation was analyzed after 6 days using alamar blue, n=3 (FIG. 4C). FIGS. 4D and 4E show knockdown of STAT3 in BAE cells. BAE cells were transfected with siRNAs targeting STAT3. qRT-PCR was performed to examine STAT3 mRNA levels. STAT3 level in siNegative was set as 1. Data from three independent experiments were averaged and are shown in FIG. 4D. In FIG. 4E, cells transfected with siRNAs were treated with LIF (10 ng/ml) and vehicle for 15 minutes. Whole-cell lysates were subjected to Western blotting with indicated antibodies. FIG. 4F shows knockdown of STAT3 abolishes LIF-induced BAE cell growth inhibition. BAE cells with STAT3 knockdown were cultured with LIF (10 ng/ml) and vehicle. Cell proliferation was analyzed after 3 days. Fluorescence reading for each vehicle group was set as 1, n=3. Bars and error bars represent mean±SD. siNegative, negative control siRNA not targeting any known genes. **, p<0.01; ***p<0.001; #, p≤0.0001; ns, not statistically significant.

FIGS. 5A-5B show that LIF does not induce vessel permeability in guinea pig skin and mouse retina. In FIG. 5A, Hairless male guinea pigs (Crl: HA-Hrhr/IAF, 450-500 g, Charles River Laboratories) were anesthetized by intraperitoneal (i.p.) administration of xylazine (5 mg/kg) and ketamine (75 mg/kg). The animals then received an intravenous injection (penile vein) of 1 ml of 1% Evans blue dye. After 15 min, intradermal injections (0.05 ml/per site) of different doses (1, 5, 25, 100, 200 ng per injection site) of rhLIF in PBS were administrated into the area of trunk posterior to the shoulder. 0.05 ml of PBS and 25 ng of VEGF in 0.05 ml of PBS were injected as negative and positive controls. 30 min after the intradermal injections, animals were euthanized by i.p. injection of pentobarbital (200 mg/kg). Skin tissues were dissected from the connective tissues and photographed, n=2. In FIG. 5B, Vascular leakage is shown in mouse retina. LIF (10 ng) or VEGF (100 ng) was injected in the vitreous cavity (0.1% BSA/PBS as control). TRITC-dextran was used to indicate the vascular leakage. Retinal vasculature was labeled by FITC-lectin, n=5.

FIGS. 6A-6F show LIF induces cell death via upregulation of cathepsin L. FIGS. 6A and 6B show LIF treatment induces cell death in BAE cells. Upon treatment with LIF (10 ng/ml) or vehicle for 24 hours, BAE cells were stained with Annexin V-Cy5. Representative images are shown in FIG. 6A. Percentages of Annexin V-positive area versus total cell-covered area were calculated and presented in FIG. 6B, n=3. FIGS. 6C and 6D show LIF induced cathepsin L expression at in BAE cells. Following treatment with LIF (10 ng/ml) or vehicle for 24 hours, qRT-PCR was performed to examine cathepsin L (CTSL) mRNA levels in BAE cells. The CTSL level in vehicle group was set as 1. CTSL mRNA levels in each sample were compared to the vehicle group and are presented as fold changes in FIG. 6C, n=3. Total proteins from LIF treated BAE cells were used for bovine cathepsin L ELISA. The cathepsin L protein levels in the vehicle-treated group were set as 1. Induction fold changes for cathepsin L protein (LIF-treated samples versus vehicle group) were calculated and fold changes from three independent experiments are shown in FIG. 6D. FIGS. 6E and 6F show Cathepsin L inhibitors CA074me and CAA0225 alleviate LIF-induced BAE cell growth inhibition. BAE cells pre-incubated with indicated concentrations of CA074me and CAA0225 for 1 hour were treated with vehicle, LIF (10 ng/ml) and VEGF (10 ng/ml). Cell growth was analyzed after 6 days, n=3. Bars and error bars represent mean±SD. *, p<0.05; **, p<0.01; ***p<0.001; #, p≤0.0001; ns, not statistically significant.

FIGS. 7A-7C show LIF induces cell cycle arrest in BAE cells. FIGS. 7A and 7B show LIF treatment reduces BrdU incorporation in BAE cells. Upon treatment with LIF (10 ng/ml) and vehicle for 48 hours, BAE cells were incubated with 10 μM of BrdU for 4 hours. Representative images of BrdU incorporation detected with an Alexa Fluor-488 conjugated BrdU antibody are shown in FIG. 7A. Percentages of BrdU positive nuclei versus DAPI-stained total nuclei were calculated and shown in FIG. 7B, n=3. FIG. 7C shows repression of cyclin A and B expression by LIF in BAE. BAE and BCE cells were treated with LIF (10 ng/ml) and vehicle for 24 hours. qRT-PCR was performed to examine CTSL1, CCNA2, CCNB1 and MYC mRNA levels. For each gene probe, the vehicle-treated group levels were set as 1. mRNA levels in LIF-treated samples were normalized to the vehicle group, n=3. Bars and error bars represent mean±SD. *, p<0.05; ***p<0.001; #, p≤0.0001; ns, not statistically significant.

FIGS. 8A-8D show effects of other IL-6 family proteins in mice eye models. Recombinant LIF (50 ng) and different doses of CT-1 in 1 μl and PBS vehicle control were injected intravitreally into mice eyes (FIG. 8A). Retinal vasculature was indicated with both live mice OCT-A imaging and CD31 immunofluorescent staining, n=5 (FIG. 8A). Retinal flat mount staining was imaged using confocal microscope (FIG. 8B). Quantification of vessels was performed using Image J. FIGS. 8C and 8D show Sodium Iodate was used to induce choroid capillary damage in mice. After sodium iodate injection, indicated amount of LIF, CT-1 or OSM was injected in eyes. Choroid capillaries were imaged under OCT-A system, n=5. Avascular area in choroid was determined and quantified using Image J.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

The present invention provides that members of the IL6 superfamily, and functional fragments thereof, can be used to increase angiogenesis in the eye of a subject in need to therapeutically treat conditions such as, but not limited to age-related macular degeneration and retinopathy of prematurity (ROP). In embodiments, the subject is a human.

In embodiments, the invention provides a method of treatment for a condition related to inadequate vascularization in the eye of a subject comprising administering to a subject in need thereof an effective amount of an IL-6 family protein, or a functional fragment thereof, to promote angiogenesis. In embodiments, the invention provides that the IL-6 family protein is leukemia inhibitory factor (LIF) or cardiotrophin-1 (CT-1).

In embodiments, the invention provides that the administration increases retinal microvessel density. In embodiments, the invention provides that the administration increases proliferation of choroidal endothelial cells. In embodiments, the invention provides that the administration stimulates angiogenesis.

In embodiments, the invention provides that the condition is age-related macular degeneration. In embodiments, the invention provides that the condition is retinopathy of prematurity (ROP).

In embodiments, the invention provides that the administration is via intravitreal injection. In embodiments, the invention provides that the effective amount does not induce vascular leakage. In embodiments, the invention provides that the effective amount does not induce edema.

In embodiments, the invention provides a method of inducing blood vessel formation in the eye of a subject comprising administering to a subject in need thereof an effective amount of an IL-6 family protein, or a functional fragment thereof.

In embodiments, the invention provides that the administration increases retinal angiogenesis. In embodiments, the invention provides that the administration increases proliferation of choroidal endothelial cells.

In embodiments, the invention provides that the subject has age-related macular degeneration. In embodiments, the invention provides that the subject has retinopathy of prematurity (ROP).

In embodiments, the invention provides that the administration is via intravitreal injection. In embodiments, the invention provides that the effective amount does not induce vascular leakage. In embodiments, the invention provides that the effective amount does not induce edema.

In embodiments, the invention provides that the IL-6 family protein is leukemia inhibitory factor (LIF). In embodiments, the invention provides that the IL-6 family protein is cardiotrophin-1 (CT-1).

### Definitions

To facilitate understanding of the invention, a number of terms and abbreviations as used herein are defined below as follows:

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or animal subject to be treated.

As used herein the term “pharmaceutical composition” refers to a pharmaceutical acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein, “effective” or “therapeutically effective” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to age-related eye diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

The term “pharmaceutically active” as used herein refers to the beneficial biological activity of a substance on living matter and, in particular, on cells and tissues of the human body. A “pharmaceutically active agent” or “drug” is a substance that is pharmaceutically active and a “pharmaceutically active ingredient” (API) is the pharmaceutically active substance in a drug.

The term “pharmaceutically acceptable” as used herein means approved by a regulatory agency of the Federal or a state government or listed in the U.S. Pharmacopoeia, other generally recognized pharmacopoeia in addition to other formulations that are safe for use in animals, and more particularly in humans and/or non-human mammals. The present invention contemplates compositions for treatment of the eye formulated for ophthalmic delivery, including intravitreal injection.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

The term “pharmaceutically acceptable salt” as used herein refers to acid addition salts or base addition salts of the compounds, such as the multi-drug conjugates, in the present disclosure. A pharmaceutically acceptable salt is any salt which retains the activity of the parent agent or compound and does not impart any deleterious or undesirable effect on a subject to whom it is administered and in the context in which it is administered. Pharmaceutically acceptable salts may be derived from amino acids including, but not limited to, cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Zurich, 2002; Berge et al., J Pharm. Sci. 66: 1, 1977). In some embodiments, a “pharmaceutically acceptable salt” is intended to mean a salt of a free acid or base of an agent or compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. An agent or compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosul fates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, [gamma]-hydroxybutyrates, glycolates, tartrates, and mandelates.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The IL-6 family of proteins for use in the present invention includes leukemia inhibitory factor (LIF) or cardiotrophin-1 (CT-1). The IL-6 family of proteins for use in the present invention can also include other IL-6 cytokines to promote angiogenesis, such as Interleukin 11 (IL-11), ciliary neurotrophic factor (CNTF), cardiotrophin-like cytokine (CLC), and Interleukin 27 (IL-27), a heterodimeric cytokine which may also be grouped in the IL-12 family. However, oncostatin M (OSM) has opposite effects. One of skill in the art can, with the knowledge of the present invention described herein, routinely screen additional IL-6 family members for angiogenic promoting activity for use in the present invention. The IL-6 family protein can be an isolated or partially purified naturally occurring protein or a recombinantly produced protein.

The amino acid sequences of such naturally occurring IL-6 family members are well-known in the art. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

In embodiments, this invention is directed to the promotion of angiogenesis for the prevention or treatment of diseases or conditions characterized by inadequate or insufficient vascularization. Such diseases or conditions include, but are not limited to, retinopathy of prematurity (ROP), age-related macular degeneration, diabetic retinopathy, glaucoma, diabetic foot ulcer, pulmonary hypertension, ischemia, chronic ulcer, baldness or hair graying, regeneration of skin flap, wound and burn healing, implantation of artificial skin, embryonic development, and preparation of blood vessels for transplantation.

This invention identifies LIF as a mitogen for primary choroidal endothelial cells. Prior to this invention, LIF had been long characterized as a negative regulator of endothelial cell growth/angiogenesis, although the exact mechanisms remained largely unknown. In 1992, LIF was reported for the first time to be an inhibitor of BAE cell growth (35). Subsequent studies described LIF also as an inhibitor of bFGF- and VEGF-induced endothelial cell proliferation (15, 41). The only exception was studies showing some mitogenic effects of LIF in immortalized endothelial cell lines generated through SV40 large T antigen (42).

This invention demonstrates, for the first time, that LIF can stimulate primary endothelial cell growth in vitro. In addition, this invention discloses that the LIF-JAK-STAT3 signaling axis is responsible for mitogenic effects in endothelial cells. Intravitreal injection of recombinant LIF significantly increases blood vessel density in adult mouse retina, confirming the proangiogenic role of LIF. Interestingly, CT-1 also induces retinal angiogenesis and is also protective in the NaIO₃ model.

In genetically engineered mouse models (GEMMs), LIF expression levels are negatively correlated with retinal vasculature development (14, 16). Yet, it has been previously reported that LIF affects multiple cell types (16, 43) and even completely disrupted retinal development in GEMMs (44). In particular, LIF negatively affected retinal astrocyte maturation and in turn promoted VEGF expression by immature astrocytes, which may contribute to increase in vessel density (16, 31, 32, 45). Therefore, alterations in retinal vasculature in the GEMMs might not be direct effects of LIF on endothelial cells. In another study, both intraperitoneal and intravitreal LIF injection led to moderate decrease in vascular density in neonatal rat eyes (22); such an inhibitory role of LIF could also be explained by its effects on retinal development. Moreover, the dose of LIF intravitreally injected was not clearly indicated in that study (22). Considering the tight, bell-shaped, dose-response disclosed in this invention, it is difficult to compare this invention to any prior studies. Indeed, at least some of the discrepancies in the literature might be explained by the widely different doses of LIF employed in different studies, ranging from a few nanograms to several hundred nanograms (16, 22).

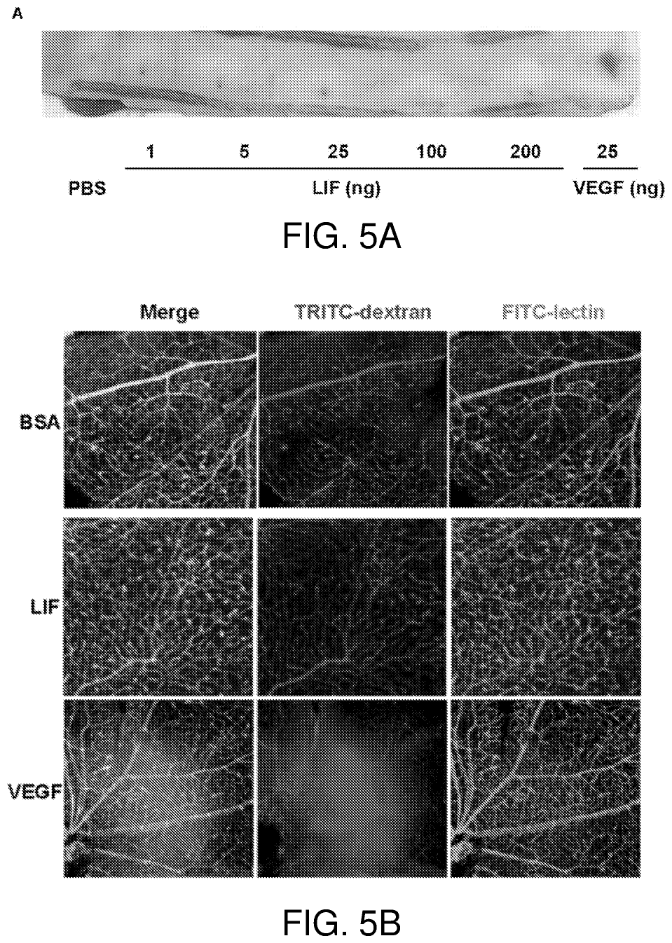

Retinopathy of prematurity (ROP) is a common blindness-causing disease among premature infants, characterized by delayed development of vasculature and regression of existing vessels, followed by hypoxia-induced retinal neovascularization (46). Drastic downregulation of VEGF expression in the eyes is associated with the onset and progression of ROP (47) and administration of exogenous VEGF alleviates the severity of ROP in mice (47). However, concerns of using VEGF as a therapeutic agent persist, since VEGF contributes to pathological neovascularization with increased vascular permeability (48). In this invention, LIF, unlike VEGF, does not induce vascular permeability in guinea pig skin (FIG. 5A). In addition, TRITC-labeled dextran was used to determine retinal microvascular leakage in mice. LIF (10 ng) or VEGF (100 ng) was injected intravitreally 15 min before TRITC-dextran injection. The result shows that unlike VEGF, LIF does not induce retinal microvascular leakage (FIG. 5B). Therefore, LIF can be used at some stages of ROP to prevent vessel regression.

Consistent with previous reports (35), this invention indicates that LIF results in BAE cell growth inhibition. This invention shows that this is attributed, at least in part, to cell death as evidenced by increase in Annexin V staining upon LIF treatment. Interestingly, two inhibitors (i.e. CA-074me and CAA0225) of lysosomal cysteine protease cathepsin L, but not caspase inhibitors, reverse LIF-induced cell death, suggesting involvement of caspase-independent cell death. Moreover, the cathepsin B-specific inhibitor CA074 fails to rescue BAE cell death, and cathepsin L, but not cathepsin B, is upregulated in LIF-treated BAE cells, indicating that cathepsin L is the executer of LIF-induced lysosomal cell death.

Induction of cathepsins B and L has been implicated in autophagy and cell death (49, 50). This invention is the first to implicate the LIF-cathepsin L pathway in induction of endothelial cell death. This raises the question whether such a signaling pathway is engaged in particular physiological or pathological processes. Interestingly, both LIF and cathepsin L have been implicated in the development and progression of vascular diseases such as abdominal aortic aneurysm and atherosclerosis (51-53). These data collectively suggest a role of the LIF-cathepsin L pathway in regulating the vasculature in pathological settings.

In this invention, LIF also leads to reduced BrdU incorporation, accompanied by decrease in cyclin A/B expression, in BAE cells, suggesting that LIF-induced cell cycle arrest plays a role in BAE growth inhibition. It was reported previously that cyclin A1 and cyclin B1 are direct STAT3 targets (54). Also, STAT3 has been implicated in both upregulation and downregulation of cyclin A/B depending on specific settings (55-58), and suppression of cyclin A expression by STAT3 was mediated by its direct target PIM1 (58). This explains why LIF represses expression of cyclin A/B in BAE cells but not in BCE cells, since induction of PIM1 by LIF is only in BAE cells.

The invention discloses opposite responses (proliferation versus growth inhibition) elicited by the same signaling pathway in two types of endothelial cells. Activated STAT3 transactivates distinct sets of genes in these two cell types. Indeed, there is differential expression of some genes upon LIF treatment in BCE and BAE cells, including downregulation of S phase and G2/M cyclin genes CCNA2 and CCNB1, as well as upregulation of lysosomal cysteine protease CTSL in BAE cells but upregulation of proliferative gene MYC only in BCE cells. Different types of endothelial cells have their unique gene expression pattern/epigenetic profiling, which determines their differential responses to the same stimulus (59-61). This invention's disclosure of opposite effects of LIF in different endothelial cells exemplifies a novel aspect of such diversity: the same signaling pathway mediates divergent effects, depending on endothelial cell-type-specific transcriptional programs. This invention reports, for the first time, that the lysosomal protease cathepsin L, induced by LIF, leads to cell death in endothelial cells.

This invention discloses, in embodiments, the unexpected mitogenic role of LIF in choroidal and retinal endothelial cells and shows that both LIF and CT-1 increases retinal microvessel density in vivo. Indeed, protecting ocular vessels such as the choriocapillaris layer in patients with wet or dry AMD is beneficial because it may prevent atrophy (62). Both LIF and CT-1 have protective effects in the NaIO₃ model suggests that these agents have therapeutic value in protecting the retinal pigment epithelium and the choriocapillaris and thus preventing atrophy in AMD. The lack of direct permeabilizing effects of LIF and likely also of CT-1 will be particularly useful in this respect. Remarkably, OSM has opposite effects, indicating a specificity in the effects of LIF and CT-1.

### EXAMPLES

### Materials and Methods

### Reagents

Antibodies: Human PDGF-AA antibody (R&D Systems, CAT #AF-221-NA), human CCL2/MCP-1 (R&D Systems, CAT #AF-279-NA), human LIF antibody (Sigma, CAT #L9277), normal goat IgG isotype control (R&D Systems, CAT #AB-108-C), and Alexa Fluor-488 conjugated BrdU antibody 3D4 (Biolegend, CAT #364106)

Small-molecule inhibitors: Baricitinib (Apexbio Technology, CAT #A414150), cobimetinib (MedChemExpress, CAT #HY-13064), BEZ235 (Selleckchem, CAT #S1409), Z-VAD-FMK (R&D Systems, CAT #FMK001), Z-DEVD-FMK (R&D Systems, CAT #FMK004), Q-VD(OMe)-OPh (Apexbio Technology, CAT #A8165), 5-AIQ hydrochloride (Sigma, CAT #A7479), CA-074 me (Calbiochem, CAT #205531), CA-074 (Tocris, CAT #4863), and CAA0225 (Calbiochem, CAT #219502)

Recombinant Proteins: Human LIF (Sigma, CAT #SRP9001), human LIF (Biolegend, CAT #593902), human PDGF-AA (Peprotech, CAT #100-13A), human Peroxiredoxin 1 (Abcam, CAT #ab74172), human IL-8 (Biolegend, CAT #574202), and human VEGF 165 (R&D Systems, CAT #293-VE)

### Cell Culture

LN-229 human glioblastoma cells were maintained in high-glucose DMEM supplemented with 5% FBS. Bovine choroidal endothelial (BCE) (P5-P9) and bovine retinal endothelial (BRE) (P5-P9) cells were maintained in DMEM-low glucose supplemented with 10% bovine calf serum (BCS), 2 mM glutamine, 5 ng/ml bFGF and 10 ng/ml VEGF on fibronectin-coated culture plates. Bovine aortic endothelial (BAE) cells (P5-P10) were maintained in DMEM-low glucose supplemented with 10% BCS. Human retinal microvascular endothelial (HRME) cells (P4-P9) were maintained in EGM2 medium with antibiotics on gelatin-coated culture plates. All cells were maintained at 37° C. in a humidified atmosphere with 5% CO2.

### Endothelial Cell Proliferation Assays

The bovine endothelial proliferation assays were performed essentially as previously described (63, 64). BCE (1×10³ cells/well) or BRE (5×10² cells/well) cells were seeded in 96-well plates in the culture medium (DMEM-low glucose supplemented with 10% BCS, 2 mM glutamine and antibiotics) plus testing materials with a total volume of 200 μl per well. BAE cells were plated in 96-well plates at a density of 2×10³ cells in the culture medium (DMEM-low glucose supplemented with 1% BCS and antibiotics) plus testing material with a total volume of 200 μl per well. HRME cells were seeded at a density of 1×10³ cells/well in gelatin-coated 96-well plates in assay medium (DMEM-low glucose supplemented with 20% FBS and antibiotics) plus testing materials to make a total volume 200 μl per well. For assays involving antibodies or small-molecule inhibitors, inhibitors or vehicle controls were first added and then test materials were added one hour later. After 6 days (unless otherwise specified), cells were incubated with alamar blue for 4 h. Fluorescence was measured at 530 nm excitation wavelength and 590 nm emission wavelength. Each experiment was carried out in duplicate/triplicate and repeated at least three times.

### LN-229 Cell Conditioned Medium

5×10⁶ LN-229 cells were seeded in a 15-cm culture dish with 35 ml of culture medium (DMEM-high glucose with 0.5% FBS and 1% antibiotics) and incubated at 37° C. for 72 h. The LN-229 CM were collected by centrifuging, filtered with a 0.22 μm filter and stored at −80° C. for later use.

### Chromatographic Enrichment of Endothelial Mitogens in LN-229 CM

Approximately 400 ml of LN-229 CM was subjected to enrichment of endothelial mitogens by sequential of chromatographic purification. CM was buffer-exchanged to 20 mM Tris, pH 8.0, filtered (0.2 μm) and loaded to a 5-ml HiTrap Q™ HP column (GE Healthcare, Pittsburgh, Pa.) using a GE AKTA Explorer System (GE Healthcare). After a stepwise elution with 0.2 M, 0.5 M, 1 M and 2 M NaCl in the Tris buffer, an aliquot of eluted fractions were tested in the BCE cell growth assay as described above. The mitogenic fractions were then pooled, diluted in 0.1% trifluoroacetic acid/H2O (TFA, ThermoFisher) and applied to a SynChropak RP C4 reverse-phase column (4.6×100 mm, Eichrom Technologies, Darien, Ill.). Fractions were eluted with a linear gradient of acetonitrile/0.1% TFA. The eluted fractions were evaporated, using a MiVac DUO Concentrator (Genevac, Ipswich, UK), washed, resuspended in PBS and tested as above. The mitogenic fractions and adjacent negative were subjected to mass spectrometry analysis.

### ELISA

VEGF and LIF levels in LN-229 CM samples were determined by a human VEGF ELISA kit (R&D Systems, CAT #DVE00) and a human LIF ELISA kit (Biolegend, CAT #443507) according to manufacturers' instructions, respectively. Cathepsin L levels in BAE cells were measured using a bovine cathepsin L ELISA kit (MyBioSource, Inc, CAT #MBS2887609) per manufacturer's instructions.

STAT3 Knockdown by siRNAs

BCE and BAE cells were plated onto 6-well culture plates at a density of 1.5×10⁵ cells/well. BCE cells were incubated in 2 ml of DMEM-low glucose supplemented with 10% BCS, 2 mM, 5 ng/ml bFGF, 10 ng/ml VEGF and antibiotics overnight, while BAE cells were cultured in 2 ml of DMEM-low glucose supplemented with 10% BCS and antibiotics overnight. 2 ml of antibiotics-free culture medium was used to replace the old medium. siRNAs, including siNegative (Ambion, CAT #AM4611), siSTAT3-915 (Invitrogen, CAT #361146C04), siSTAT3-1492 (Invitrogen, CAT #361146C05) and siSTAT3-454 (Invitrogen, CAT #384235A10), were mixed with Lipofectamine RNAiMAX reagent (ThermoFisher Scientific, CAT #13778150) in Opti-MEM™ I Reduced Serum Medium (Gibco, CAT #31985062) according to manufacturer's instructions. Briefly, a mix containing 25 pmol of siRNA, 7.5 ul of RNAiMAX reagent and 125 ul of Opti-MEM medium was used to transfect cells in each well, which makes a final siRNA concentration of 12.5 nM. A mix of RNAiMAX and Opti-MEM was used as no siRNA control. Cells were incubated with siRNAs for 8 hours and then fresh normal culture medium was used to replace the siRNA-containing medium. 24 hours after transfection with siRNAs, cells were used for endothelial proliferation assays and RNA/protein extraction.

### Western Blotting

BCE and BAE cells were cultured in growth medium overnight. Growth medium was removed and then cells were washed twice with PBS. Recombinant human LIF was added to cells for 15 minutes, following 3-hour incubation in the following medium: DMEM-low glucose supplemented with 10% BCS, 2 mM glutamine and antibiotics for BCE cells, and DMEM-low glucose supplemented with 1% BCS and antibiotics for BAE cells. If applicable, small-molecule inhibitors (i.e. baricitinib, cobimetinib, BEZ235 and vehicle control DMSO) were added to the cells 1 hour prior to LIF treatment. Cells were then lysed with RIPA lysis buffer (Life Technologies, CAT #89901) plus protease and phosphatase inhibitor cocktail (ThermoFisher Scientific CAT #78440). Protein concentrations in cell lysates were measured with the BCA assay (ThermoFisher Scientific CAT #23227). Equal amount of proteins were subjected to electrophoresis in NuPAGE 4-12% Bis-Tris gels (ThermoFisher Scientific, CAT #NW04125BOX) and then transferred onto PVDF membranes. Membranes were blocked with 5% non-fat milk in TBST at room temperature for 1 hour, incubated with primary antibodies indicated below in TBST containing 0.5% non-fat milk at 4° C. overnight then with secondary HRP-conjugated antibodies (1:2000, GE Healthcare) at room temperature for 1 h. Signals were developed with SuperSignal™ West Pico PLUS Chemiluminescent Substrate (ThermoFisher Scientific). Primary antibodies used: anti-phospho-STAT3 (Cell Signaling, CAT #9131, 1:3000), anti-STAT3 (Cell Signaling, CAT #4904, 1:3000), anti-phospho-ERK (Cell Signaling, CAT #4376, 1:5000), anti-ERK (Cell Signaling, CAT #4695, 1:5000), anti-phospho-AKT Ser473 (Cell Signaling, CAT #4060, 1:2000), anti-AKT (Cell Signaling, CAT #4691, 1:2000) and HRP-conjugated anti-beta-actin (Sigma, CAT #AC-15, 1; 10000).

RNA Extraction and qRT-PCR

BCE and BAE cells, after the indicated treatments, were lysed with Trizol reagent (Invitrogen, CAT #15596026) and subjected to RNA extraction following manufacturer's instructions. RNA concentrations were determined with Nanodrop 2000 (ThermoFisher Scientific) and 1 μg of total RNAs were reverse-transcribed to cDNAs using the High-Capacity cDNA Reverse Transcription Kit (Applied Biosystems, CAT #4368814). Equal amounts (generally 10 ng/reaction) of cDNAs were subjected to qRT-PCR analyses using the TaqMan Fast Advanced Master Mix (Applied Biosystems, CAT #4444557) and the ViiA7 Real-time PCR system. Relative mRNA levels of the examined genes were normalized to the internal control RPLP0 (Ribosomal Protein Lateral Stalk Subunit P0), determined by comparing with control sample group, and reported as fold changes. TaqMan gene expression assay probes were used: bovine RPLP0 (Bt03218086_ml), bovine STAT3 (Bt03259865_ml), bovine CTSL1 (Bt03257307_ml and Bt03257309_ml), bovine CTSB (Bt03259161_ml), bovine MYC (Bt03260377_ml), bovine JunB (Bt03246919_s1), bovine CCNA2 (Bt03240503_g1), bovine CCNB1 (Bt03237853_g1), and bovine PIM1 (Bt03212957_ml). The experiment was carried out in triplicate and repeated three times.

### Annexin V Staining for Cell Death

BAE cells were plated at a density of 2×10⁴ cells/well with 1 ml of culture medium (DMEM-low glucose plus 10% BCS) in 12-well plates and then incubated at 37° C. overnight. After removal of culture medium, cells were incubated in 0.5 ml of DMEM-low glucose plus 1% BCS. LIF (10 ng/ml) and vehicle control (0.1% BSA in PBS) were added to the cells. Following LIF treatment for 24 hours, cells were examined for cell death marker Annexin V using Annexin V-Cy5 Apoptosis Staining Detection Kit (Abcam, CAT #ab14150) according to manufacturer's instructions. Briefly, cell culture medium was removed and 0.5 ml of Annexin V binding solution was laid over onto the cells. Cells were incubated at room temperature for 5 min following addition of 5 μl of Annexin V-Cy5. Then, the staining solution was discarded and replaced with 0.5 ml of Annexin V binding solution. Imaging of Annexin V staining were performed using Keyence Microscope BZ-X710 (Keyence Corporation, Osaka, Japan). Four random fields were selected and the percentages of Annexin V-staining area in total cell-covered area as indicatives for cell death were determined using ImageJ software. Imaging of Annexin V staining were performed using Keyence Microscope BZ-X710 (Keyence Corporation, Osaka, Japan). The experiment was carried out in triplicate and repeated three time.

### BrdU Incorporation Assay

BAE cells were plated at the density of 2×10⁴ cells/well with 1 ml of culture medium (DMEM-low glucose plus 10% BCS) in a 12-well plate with a 18-mm poly-D-lysine-treated coverslip in each well, incubating at 37° C. overnight. After removal of culture medium, cells were incubated in 0.5 ml of DMEM-low glucose plus 1% BCS. LIF (10 ng/ml) and vehicle control (0.1% BSA in PBS) were added to the cells. Upon LIF treatment for 48 hours, cells were subjected to BrdU incorporation by adding 2.5 μl of 2 mM BrdU in DMSO to each well to a final concentration 10 μM and incubating for 4 hours. Then, cells are subjected to BrdU immunofluorescence staining using an antibody against BrdU conjugated with Fluor alexa-488 (Biolegend, CAT #364106, 1:400). Briefly, BrdU labeling medium was removed from the culture plates and cells were fixed with 3.7% formaldehyde in PBS at room temperature for 15 minutes. Cell DNAs were denatured with 1N HCl on ice for 10 minutes and 2N HCl at room temperature for 10 minutes following cell permeabilization with 0.1% Triton X-100 in PBS (PBST). Cell coverslips were incubated with fluor alexa-488 conjugated BrdU antibody in 5% goat serum-PBST overnight at 4° C. Then, coverslips were mounted to glass slides with Fluoroshield Mounting Medium With DAPI (Abcam, CAT #ab104139). Imaging of BrdU staining were performed using Keyence Microscope BZ-X710 (Keyence Corporation, Osaka, Japan). Four fields were randomly selected for each sample and the BrdU-positive nuclei as well as total nuclei (DAPI-positive) were counted manually; the percentages of BrdU-positive cells were determined by dividing the numbers of BrdU-positive nuclei with the numbers of total nuclei. The experiment was carried out in duplicate/triplicate and repeated three times.

### Mouse Choroidal Explant Assay

In a 48-well plate, 60 μL of growth factor-reduced basement membrane extract (GFR-BME) (Corning, CAT #354230) was added to each well and allowed to solidify at 37° C. for 20 minutes. A dice of the peripheral choroid-scleral complex (approximately 1 mm×1 mm), dissected from male C57BL/6J mice (age P20), was added to the center of each well as previously described (23). A top layer of 60 μL GFR-BME was added to each well, followed by incubation at 37° C. for 30 minutes. Upon adding 500 μL of endothelial cell growth basal medium EBM-2 (Lonza, CAT #CC3156) supplemented with 2% FBS and antibiotics, endogenous VEGF activity of choroid explants was blunted by 5 μg/ml of anti-VEGF Mab B20-4.1.1. After 90 minutes of incubation with the antibody, 10 ng/ml of LIF or PBS control was added in the test wells. Tissues were incubated in standard cell culture conditions with 5% CO2 and fresh media were changed every 48 hours. Phase contrast Z-stack images of each explants were taken on day 5 using a Keyence microscope. Vessel sprouting areas were quantified using ImageJ software. The experiment was repeated three times and data were obtained by analyzing 5 replicates per each condition each time.

### Intravitreal Injection of Recombinant Proteins in Mouse Eyes

Male C57BL/6J mice (6-8 week and P5) were anesthetized with ketamine/Xylazine cocktail. The indicated amounts of recombinant LIF (Sigma, CAT #SRP9001) in 1 μl of PBS and PBS vehicle control were injected intravitreally with a 33-gauge Hamilton syringe. Seven (for adult mice) or 3 (for neonatal mice) days after injection, animals were euthanized, eyes were then enucleated and fixed in 4% paraformaldehyde (PFA) for 15 min. Choroid-sclera complexes and retinas were separated and anti-CD31 immunofluorescence (IF) or lectin labeling was performed to evidence the vasculature by whole mount staining of both retina and choroidal tissues or flat-mounts of retina. For CD31 IF, rat anti-mouse antibody (BD Biosciences, CAT #550274) was diluted 1:100 and incubated overnight at 4° C. After 4-hour incubation with the Alexa Fluor-488-conjugated anti-rat antibody (Life Technologies, CAT #A11006), whole mounts were imaged via the 488 nm channel using Keyence Microscope BZ-X710 (Keyence Corporation, Osaka, Japan) or A1R Confocal STORM super-resolution system (Nikon). For lectin staining, Dylight-488-labeled lectin (Vector Laboratories, CAT #DL-1174) was diluted at 1:200 and images were obtained using A1R Confocal STORM super-resolution system (Nikon). For Quantification of vascular density in choroids and retina was carried out by Image J. Student's t test was used for statistical analysis. Each experiment was repeated three times with similar results each time, and each treatment group consists of 4 or 5 individual samples in every experiment. All animal experimental procedures were approved by the Institutional Animal Care and Use Committee (IACUC) of the University of California San Diego and conducted in accordance with the guidelines of the Animal Care Program (ACP).

### Sodium Iodate Model

Eight-week-old C57BL/6J mice were anesthetized with ketamine/xylazine cocktail. Sterilized NaIO₃ was administered as a single intravenous injection (20 mg/kg body weight) (28) (29). Control mice were injected with PBS. PBS, LIF (50 ng), CT-1 (different doses) or OSM (10 ng) was injected intravitreally in five-mice groups. Five, seven and nine days after injection, choroid capillaries were monitored by OCT-A system. 9 days after injection, mice were killed and eyes were harvested for H&E and immunofluorescent staining. Avascular area in choroid capillaries was analyzed using ImageJ.

### Measurement of Vascular Leakage in Retina

Recombinant human VEGF (100 ng) or LIF (10 ng) was injected into the vitreous (0.1% BSA PBS solution as vehicle control). TRITC-dextran (50 mg/ml, 100 ul) was then injected into the tail vein. Ten minute later, animals were sacrificed and eyes were enucleated. Retina flat mount was imaged under microscope (65).

### Optical Coherence Tomography Angiography (OCTA) Imaging

Optical coherence tomography angiography (OCTA) imaging of the retina of adult mice was performed 7 days after LIF injection, using a 1300 nm optical coherence tomography (OCT) system developed by Dr. R. K. Wang's group at University of Washington Seattle, in agreement with previously described methodology (66). Briefly, the swept laser operated in single longitude mode with a 90 nm bandwidth centered at 1300 nm and 200 kHz A-line rate was used to scan mouse retina and to generate images of vasculature in a field of view of 1.5×1.5 mm². 2500 B-frames were captured at 500 cross-sections with five repeated B-frames at each cross-section. To quantify the retinal vascular density, retinal and choroidal layers in 3D structure OCT scans were separated by the hyper-reflecting retinal pigment epithelium (RPE). Then the en face maximum intensity projection was generated. The vessel density was then determined by calculating the percentages of vessel-covered area in total area of view using the ImageJ software.

### Statistical Analysis

Experiments were repeated at least three times with similar results except for the mass spectrometry analyses. Bar charts represent mean±standard deviation (sd). For comparison between the only two groups in a study, two-tailed Student's t test was performed. For comparisons among groups in a study with more than two groups of data, one-way ANOVA with multiple-comparison were performed. For comparisons among groups in a study with two or more variables, two-way ANOVA with multiple-comparison were performed. p<0.05 was deemed as statistically significant. All statistical analyses were performed use Graphpad Prism software package.

### Results

### Identification of LIF as a Mitogen for Choroidal Endothelial Cells

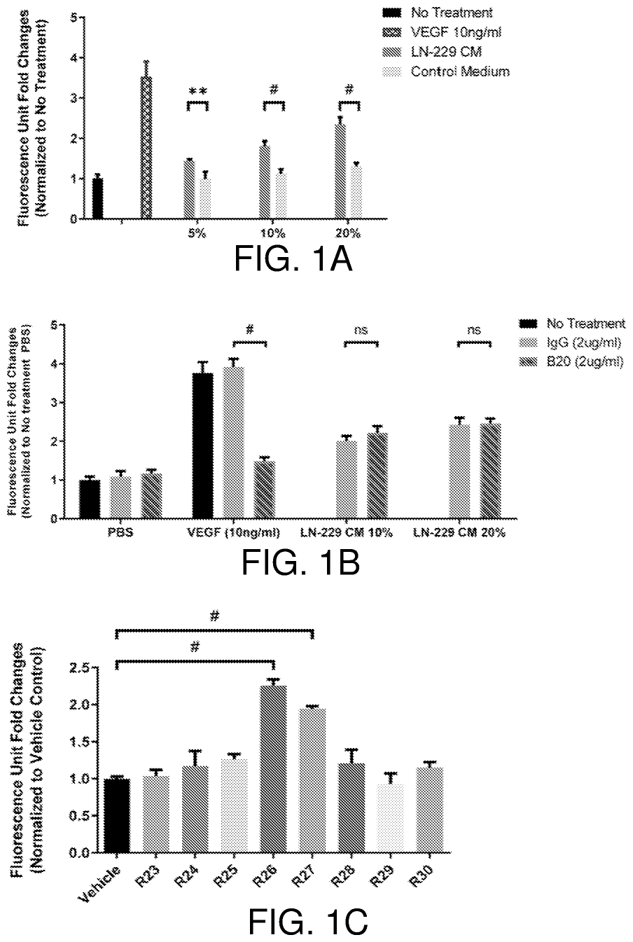

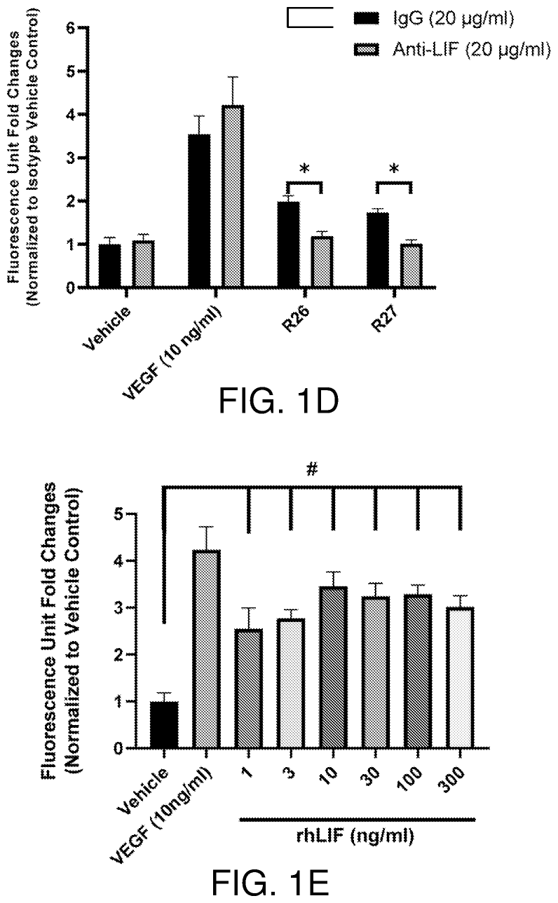

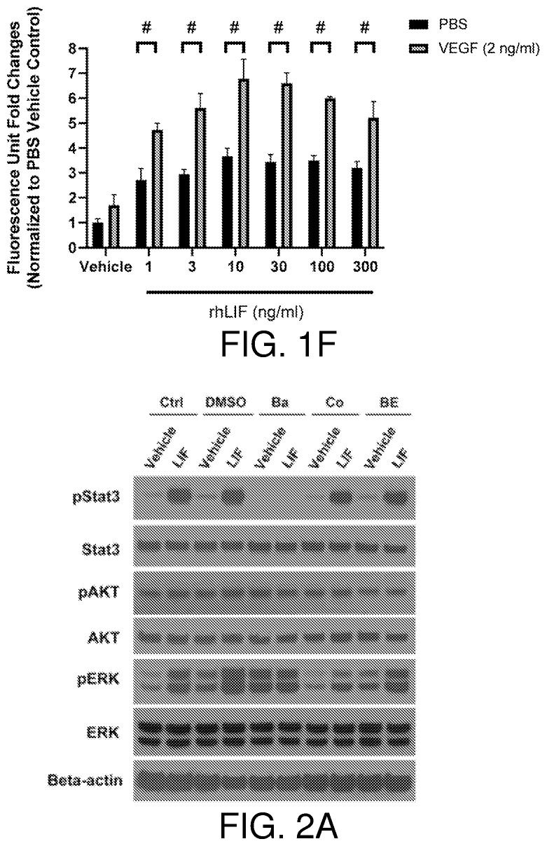

The LN-229 cell conditioned medium (LN-229 CM) is able to stimulate growth of bovine choroidal endothelial (BCE) cells (FIG. 1A). However, in agreement with previous studies (9, 10), LN-229 cells secrete very little VEGF in the medium. The anti-VEGF antibody B20-4.1 (11) does not suppress the mitogenic effects of LN-229 CM (FIG. 1B), suggesting the involvement of VEGF-independent pathways. The angiogenic factor profile of LN-229 CM was examined using specific antibody arrays. This analysis reveals that the majority of known angiogenic factors are undetectable, except PDGF-AA, CCL2 (also known as MCP-1) and interleukin 8 (IL-8), which were abundant in the CM. However, antibodies neutralizing PDGF-AA or CCL2 failed to suppress BCE cell growth induced by LN-229 CM. Moreover, recombinant PDGF-AA and IL-8 fail to stimulate BCE cell growth (Table 1).

TABLE 1
Recombinant human PDGF-AA, IL-8 and PRDX1 do not stimulate
BCE cell growth in vitro. BCE cells were treated with the
indicated concentrations of PDGF-AA/IL-8/PRDX1. Cell
growth was determined at day 6. Cell growth in each treatment
group was normalized to the vehicle control group, n = 3.
ns, not statistically significant.

| | 1 | 3 | 10 | 30 | 100 | 300 | 1000 |
|---|---|---|---|---|---|---|---|
| | ng/ml | ng/ml | ng/ml | ng/ml | ng/ml | ng/ml | ng/ml |
| PDGF-AA | ns | ns | ns | ns | ns | ns | N/A |
| IL-8 | ns | ns | ns | ns | ns | ns | N/A |
| PRDX-1 | N/A | ns | ns | ns | ns | ns | ns |

To identify mitogenic factor(s) in the LN-229 CM, a proteomic approach was taken. The BCE mitogenic activity was enriched through two sequential chromatographic steps, anion-exchange and reverse-phase chromatography. At each step, only one peak of absorbance, composed of 4 to 5 contiguous fractions, showed mitogenic activity. After the reverse-phase column step, the peak mitogenic fractions (R26 and R27), the minimally mitogenic (R25 and R28), and adjacent negative (R24 and R29) fractions (FIG. 1C) were subjected to mass spectrometry analyses. A short list of 5 candidate proteins was generated by screening out intracellular proteins (Table 2).

TABLE 2
Candidate proteins generated from Mass-spectrometry analysis of LN-
229 CM reverse-phase fractions. Candidates were identified by excluding
intracellular proteins and proteins showing higher abundance in
inactive fractions compared to those in mitogenic factions. Proteins
were ranked for relative abundance as described in Methods.

| Ranking | Protein Identity |
|---|---|
| 1 | PRDX1_HUMAN Peroxiredoxin-1 |
| 2 | PRDX2_HUMAN Peroxiredoxin-2 |
| 3 | PRDX6_HUMAN Peroxiredoxin-6 |
| 4 | LIF_HUMAN Leukemia inhibitory factor |
| 5 | A2MG_HUMAN Alpha-2-macroglobulin |

Four of the 5 proteins listed were serum components and functioned as redox enzymes, including peroxiredoxin (PRDX)-1, -2 and -6 as well as alpha-2-macroglobulin, while LIF stood out as a cytokine. LIF, a member of the interleukin 6 (IL-6) family proteins, is broadly expressed and exerts effects in multiple cell types and tissues, and has been implicated in various critical physiological processes including embryonic stem cell self-renewal, blastocyst implantation, astrocytes differentiation (12, 13). The presence of LIF herein was unexpected, since this cytokine had been previously characterized as an endothelial cell growth inhibitor and an anti-angiogenic agent (14-16). However, an antibody directed against LIF completely suppressed growth of BCE cells induced by the reverse phase fractions (FIG. 1D). The LIF levels in each fraction were strongly correlated with mitogenic activity: the most bioactive fractions, R26 and R27, showed the highest LIF concentrations, R25 and R28 had trace amounts of LIF, while the inactive fraction R24 and R29 were devoid of LIF (Table 3).

TABLE 3
LIF concentrations (ng/ml) in mitogenic fractions
(R26 and R27) and in adjacent negative fractions
(R24, R25, R28 and R29) from reverse phase chromatography
were measured with a human LIF ELISA kit.

| Fraction No. | R24 | R25 | R26 | R27 | R28 | R29 |
|---|---|---|---|---|---|---|
| LIF | 0.2 | 5.9 | 72.9 | 44.8 | 4.9 | 0 |
| concentration | | | | | | |
| (ng/ml) | | | | | | |

These observations suggested that LIF might be responsible for the mitogenic effects. Indeed, recombinant LIF stimulated growth of BCE cells (FIG. 1E), while the other candidate, PRDX1, had no effect (Table 1), further confirming LIF as the mitogenic factor. When tested on bovine retinal endothelial (BRE) cells, LIF also exerted mitogenic activity. Interestingly, VEGF and LIF together resulted in greater than additive mitogenic effects in both BCE (FIG. 1F) and BRE cells, suggesting a synergistic relationship between LIF and VEGF. Indeed, although LIF did not elicit a strong mitogenic response in human retinal microvascular endothelial cells, its addition significantly enhanced VEGF-stimulated growth.

### Effects of LIF on Endothelial Cell Growth are Mediated by the JAK-STAT3 Pathway

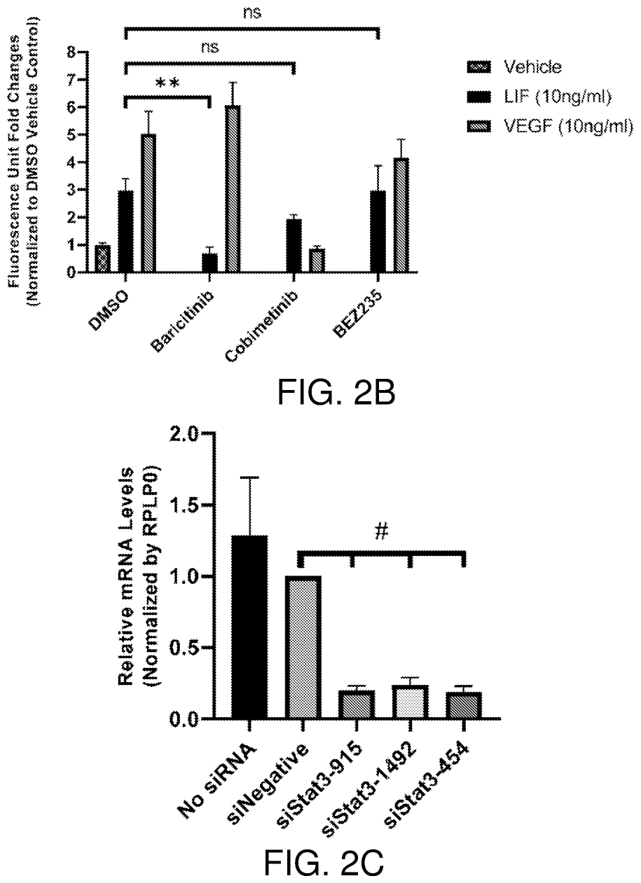

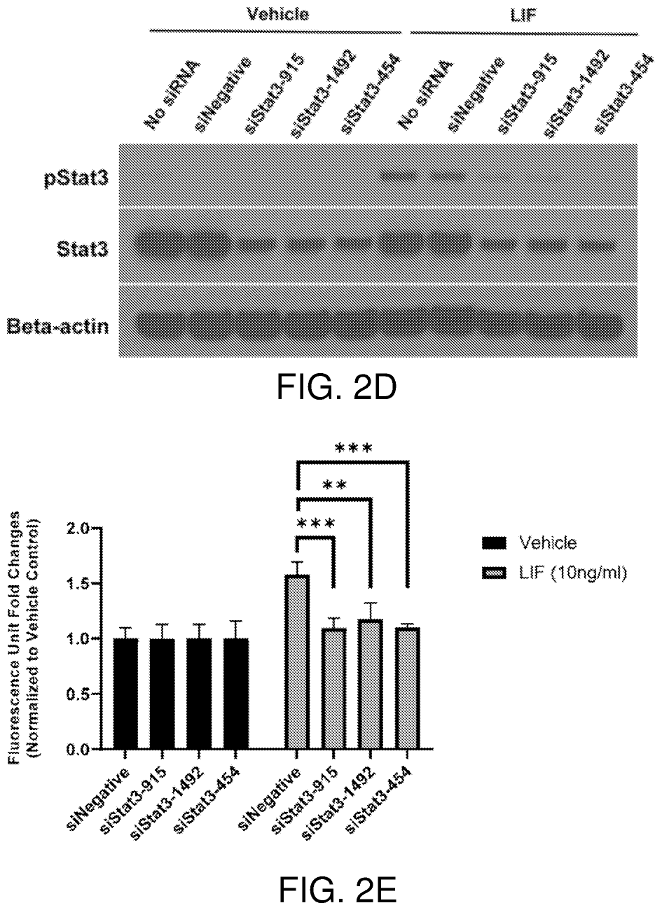

Although all members of the IL-6 family share a receptor component, gp130, LIF signaling transduces via the gp130:LIFR receptor dimer, while IL-6 activates its downstream signal through the IL6Rα:gp130:gp130:IL6Rα tetramer (12). Among four Janus kinases (JAK1, JAK2, JAK3 and TYK2) associated with gp130, LIF signaling selectively activates JAK1 through transphosphorylation (12, 17, 18). Upon activation by LIF, JAKs elicit three distinct signaling cascades: JAK-STAT, PI3K-AKT-mTOR and RAS-MAPK, which contribute to different functions in a cell type specific manner (12, 19). As to JAK-STAT pathway, LIF signaling preferentially activates STAT3 though STAT1 and STATS can also be phosphorylated by JAK1 (19, 20). To examine which pathways are responsible for LIF-induced growth stimulation in BCE cells, a set of small-molecule inhibitors baricitinib, cobimetinib and BEZ235, which are specifically against JAK1/2, MEK1/2 (MAPK pathway) and PI3K/mTOR, respectively, were employed. In BCE cells, LIF treatment for 15 minutes elicited phosphorylation of STAT3 and ERK but showed little effects on AKT phosphorylation (FIG. 2A). Pre-incubation with the JAK1/2 inhibitor baricitinib almost completely suppressed LIF-induced phosphorylation of STAT3 and ERK MAPK (FIG. 2A), while cobimetinib pretreatment blocked ERK phosphorylation but showed no effects on STAT3 and AKT phosphorylation (FIG. 2A). BEZ235 had only moderate effects on AKT phosphorylation regardless of LIF treatment (FIG. 2A). Moreover, baricitinib completely blocked LIF-induced cell growth, while cobimetinib showed minimal effects and the PI3K/mTOR inhibitor BEZ235 had no effect on LIF-stimulated cell growth (FIG. 2B). These observations suggest that the MAPK and PI3K pathways might not be major contributors to LIF stimulation in BCE cells, and thus JAK-STAT is implicated. Since STAT3 is the preferential mediator in LIF-induced JAK-STAT signaling cascade (19, 20) and has been implicated in proliferation and survival in a wide variety of cell types (21), the role of STAT3 in BCE by siRNA knockdown was further examined. siRNAs successfully dampened STAT3 levels at both RNA and protein levels in BCE cells (FIGS. 2C and 2D). Downregulation of STAT3 blocked LIF-induced BCE cell growth in vitro (FIG. 2E). These observations indicated that the JAK-STAT3 signaling axis mediated the mitogenic effects of LIF in BCE cells.

### LIF Promoted Endothelial Cell Growth Ex Vivo and In Vivo

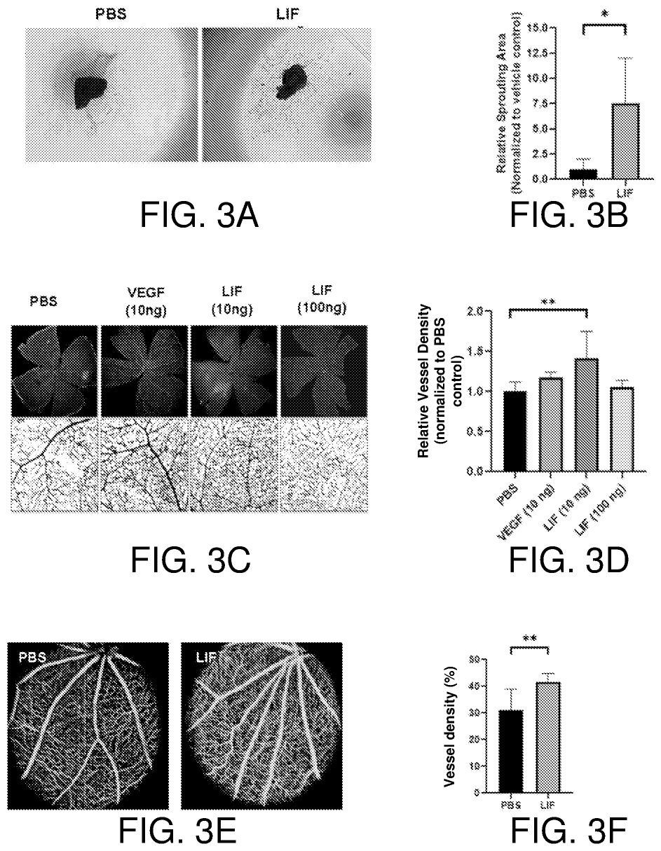

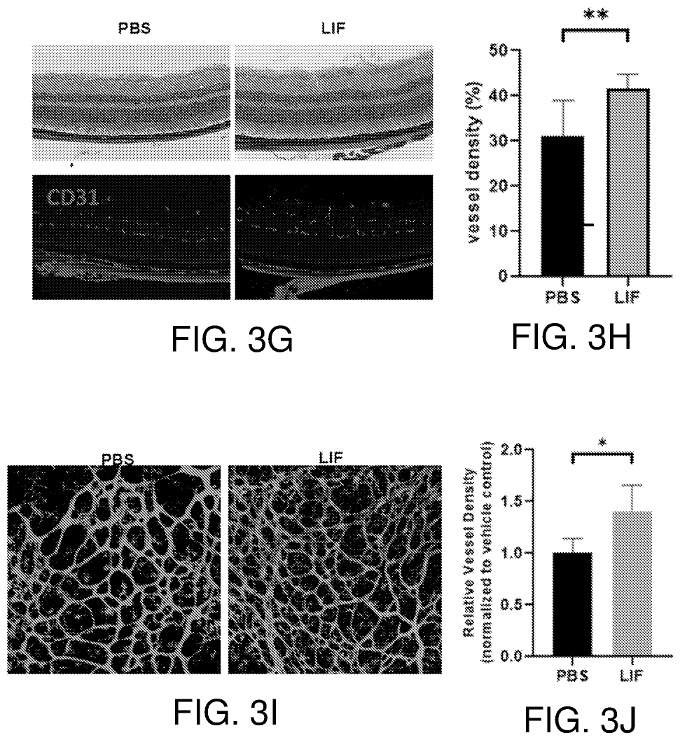

LIF can induce proliferation of choroidal and retinal endothelial cells in vitro. However, previous reports had suggested that LIF could negatively affect vessel functions in developing eyes (14, 16, 22). To resolve these apparent discrepancies, whether LIF functions differentially in endothelial cells ex vivo and in vivo, especially in the eyes, was examined. The effects of LIF on choroidal endothelial cells were first examined in an ex vivo choroidal explant model modified from a previous report (23). In response to LIF, microvascular outgrowth from the explant into the matrigel was significantly enhanced compared with that in the control (FIGS. 3A and 3B). Next, LIF effects in vivo were examined by intravitreal injection in 6-8 week old mice. Administration of LIF at the dose of 10 ng per eye significantly increased retinal microvessel density, as assessed by immunohistochemistry (IHC) with an antibody against endothelial cell surface marker CD31, while the dose of 100 ng was less effective (FIGS. 3C and 3D), consistent with the bell-shaped responses observed for many cytokines (24). Optical coherence tomography angiography (OCTA) also documented significant increases in retinal vessel density following LIF injection (FIGS. 3E and 3F). Immunofluorescence staining for CD31 in cross sections of mouse eyes also demonstrated that LIF injection increased vessel density in adult mouse retina (FIGS. 3G and 3H).

To verify that such proangiogenic effects were truly induced by LIF rather than by trace amount of contaminants such as endotoxin or by unspecific events related to the injection, recombinant LIF was heat-inactivated by exposure to 95° C. for 2 hours, which does not affect endotoxin stability (30). Such treatment abolished LIF ability to promote mitogenesis in vitro and angiogenesis in vivo. However, a previous study using LIF knockout mice suggested that LIF expression was negatively associated with retinal vessel density (16). The difference between such an observation and this invention's data raises the possibility that LIF performs distinct roles in regulating retinal angiogenesis at different developmental stages. Importantly, LIF also plays a critical role in retinal astrocyte maturation, which may secondarily affect development of retinal vasculature (31, 32). To examine the effects of LIF on the developing retinal vasculature and to minimize its impact on astrocyte development, LIF was intravitreally injected into 5-day postnatal (P5) mice in which retinal vasculature is developing but the astrocyte network has already established and is undergoing maturation (33, 34). LIF treatment in such neonatal mice also resulted in significant increase in vascular density as assessed three days after the injection (FIGS. 3I and 3J), confirming the pro-angiogenic effects of LIF in the retinal vasculature.

Since LIF is a member of the interleukin-6 (IL-6) family (25), the effects on retinal vascularization of two other family members, cardiotrophin-1 (CT-1) (26) and oncostatin M (OSM) (27) were tested. Comparable to 50 ng LIF, 20 ng and 100 ng CT-1 resulted respectively in approximately 30% and 50% increases in retinal density. However, instead of promotion, vascular density decreased in the retina of OSM-treated mice. The different effects induced by OSM from that of LIF and CT-1 on the retinal vasculature suggest that OSM may not activate the same signaling pathway as LIF and CT-1 do, for OSM can bind to both gp130::LIFR and gp130::OSMR receptor complexes, while LIF and CT-1 only utilize gp130::LIFR complex.

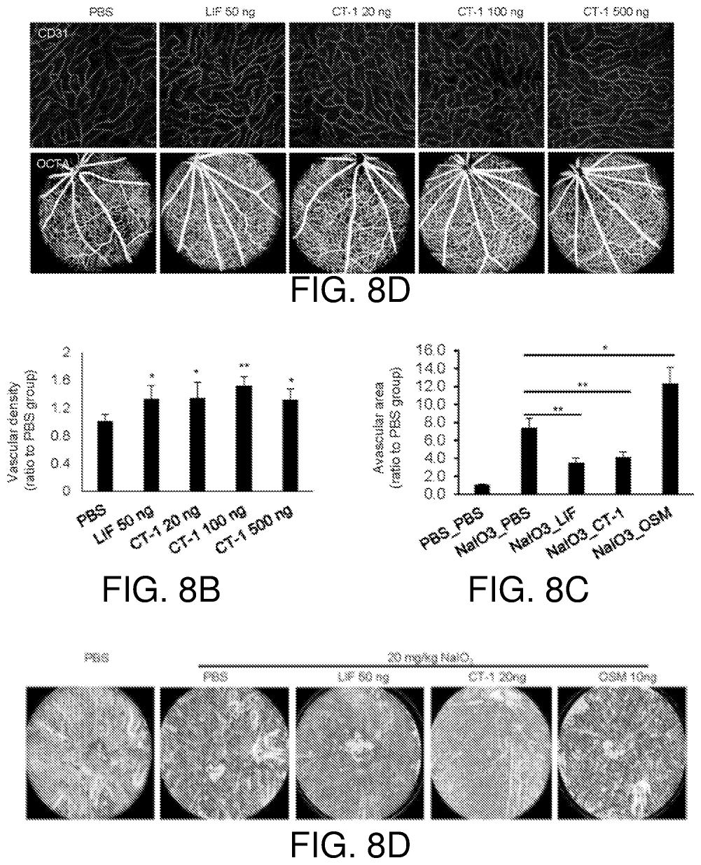

The NaIO₃ mouse model has been widely used as a pre-clinical model of atrophic AMD (28). In this model, both RPE layer and choroid capillaries are heavily damaged (29). Therefore, LIF, CT-1 and OSM were tested for their ability to promote choroid capillary recovery in this model. After intravenous injection of NaIO₃, LIF, CT-1 or OSM were injected intravitreally. In agreement with the effects on the retinal vasculature, LIF and CT-1 reduced avascular areas compared to PBS group. In contrast, avascular areas in OSM-treated choroids were larger than in the PBS group (FIGS. 8C and 8D). The protective effects of LIF and CT-1 on the retinal vasculature against NaIO₃ treatment may be attributed to both their direct mitogenic activities in retinal endothelial cells and potentially also to their ability to protect retinal RPE cells from oxidative stress-induced damages, which in turn supports maintenance of the retinal vasculature via secretion of proangiogenic factors, e.g. VEGF.

### LIF Conferred Growth Inhibition Via the JAK-STAT3 Pathway

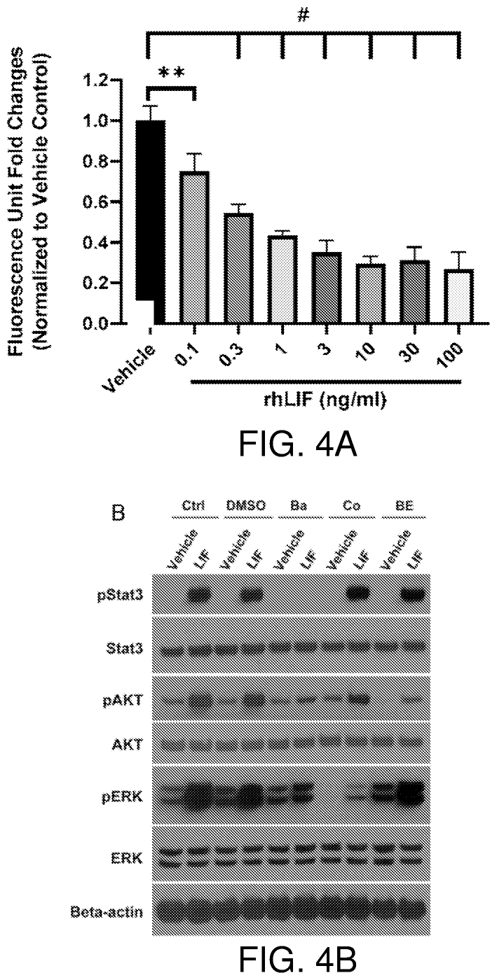

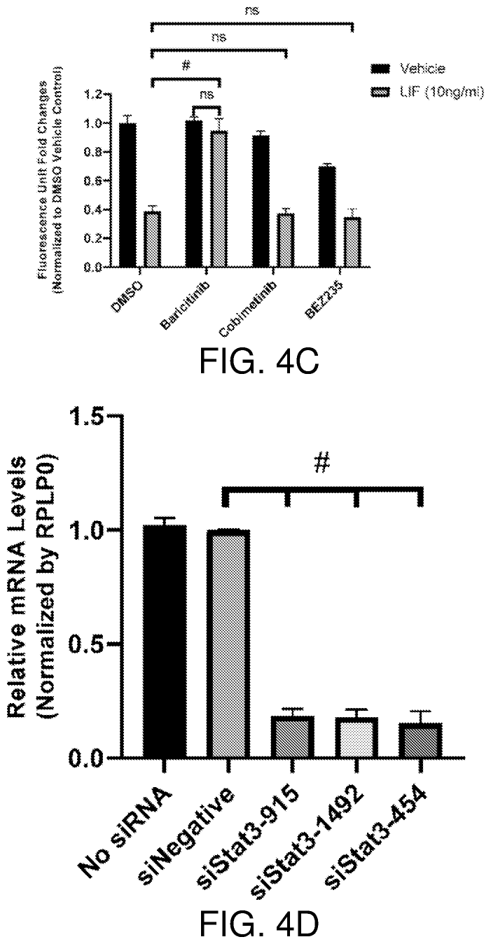

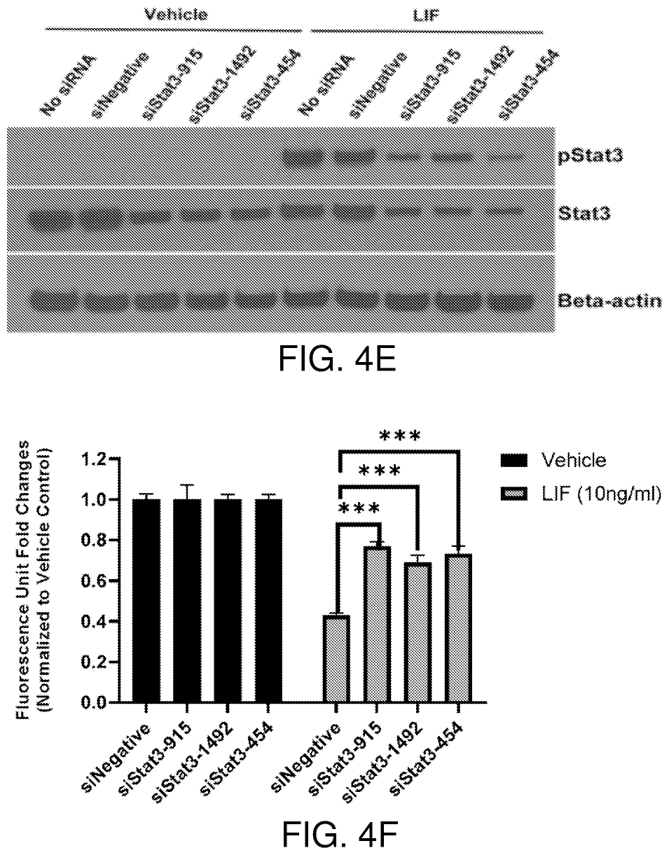

In agreement with previous studies (35), LIF resulted in growth inhibition of BAE cells (FIG. 4A), suggesting a complex role of LIF in regulating endothelial functions. To interrogate the LIF-induced signaling cascade in BAE cells, baricitinib, cobimetinib and BEZ235 were used to inhibit LIF-gp130:LIFR downstream components JAK1/2, MEK1/2 and PI3K/mTOR. In BAE cells, LIF treatment for 15 minutes led to phosphorylation of STAT3, ERK (MAPK) and AKT (FIG. 4B). Baricitinib pretreatment significantly suppressed LIF-induced phosphorylation of STAT3, ERK and AKT, while cobimetinib and BEZ235 pretreatment also effectively repressed phosphorylation of ERK and AKT, respectively (FIG. 4B). Interestingly, baricitinib was the only inhibitor that reversed growth suppression induced by LIF in BAE cells (FIG. 4C), suggesting that the JAK-STAT pathway mediated effects of LIF in BAE cells. To further examine whether inhibition of BAE cells by LIF was attributed to the JAK-STAT3 cascade, STAT3 was knocked down by approximately 80% with 3 different siRNA in BAE cells (FIGS. 4D and 4E). Intriguingly, knockdown of STAT3 in BAE cells ameliorated growth inhibition by LIF (FIG. 4F). These observations demonstrate that the LIF-JAK-STAT3 signaling pathway could play opposite roles in regulation of endothelial cell growth, which was dependent on the types of endothelial cells.

### LIF Inhibited BAE Cells Growth Via Cathepsin L-Dependent Cell Death and Cell Cycle Arrest

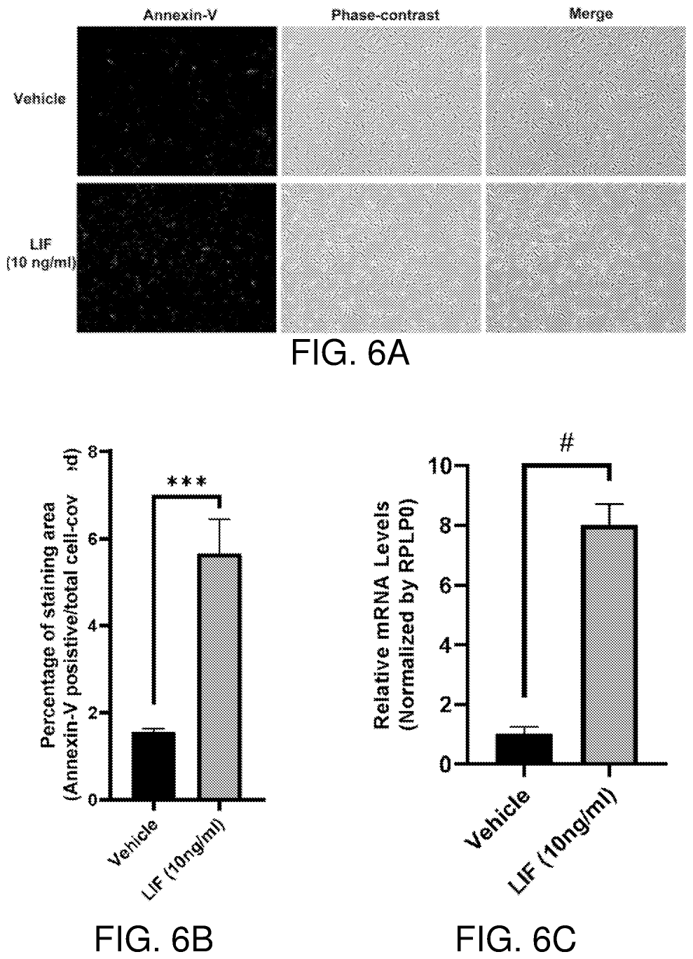

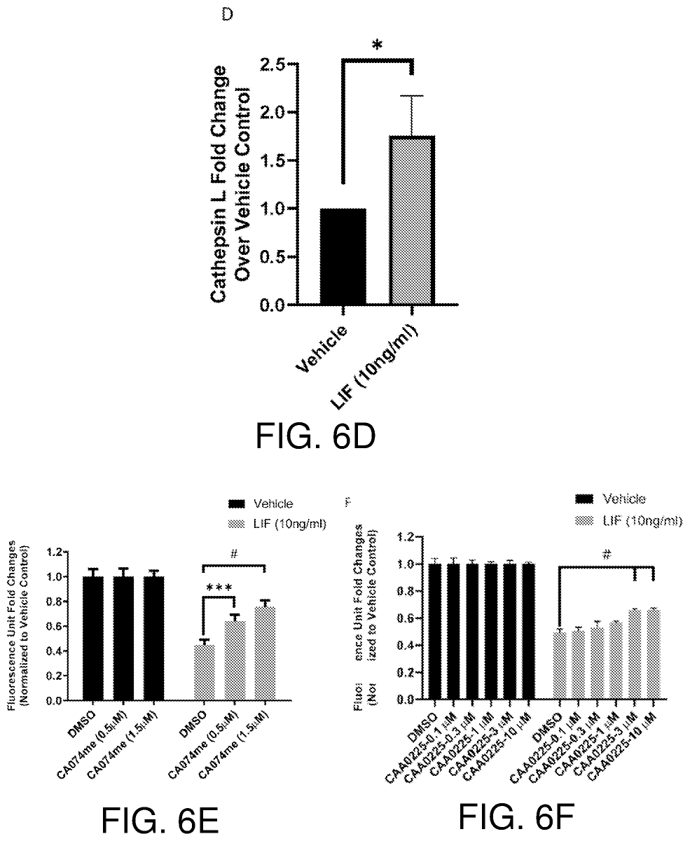

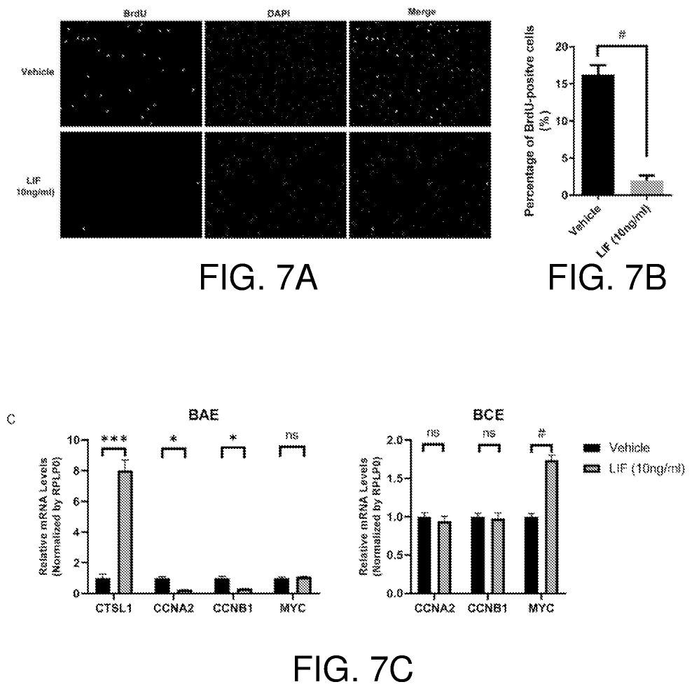

Which growth inhibitory effects (e.g. cell cycle arrest, cellular senescence or programmed cell death) were induced by LIF in BAE cells was examined next. Since IL-6-STAT3 signaling was tightly associated with cellular senescence (36-38), it was first hypothesized that LIF-STAT3 axis also induced senescence in BAE cells. However, in the senescence-associated-β-galactosidase assay, increased numbers of senescent cells in BAE cell treated with LIF for 48 hours was not observed, suggesting that senescence was not the main effect elicited by LIF in BAE cells. Interestingly, staining for the cell death marker Annexin V showed an increased proportion of cells were Annexin V positive in BAE cells treated with LIF for 24 hours (FIGS. 6A and 6B), indicating that LIF treatment induced cell death. Surprisingly, co-incubation with the caspase inhibitors (Q-VD-OPH, Z-VAD-fmk and Z-DEVD-fmk) or poly(adenosine 5′-diphosphate ribose) polymerase (PARP) inhibitor (5-AIQ) was not able rescue the cell death phenotype induced by LIF. These data suggest that a caspase-independent pathway may be involved in LIF-mediated cell death in BAE cells. To investigate the molecular basis for differentiated roles of LIF in BAE and BCE cells, genes induced/suppressed by LIF were analyzed by RNA-seq in BAE and BCE cells incubated with LIF for 6 hours. Remarkably, LIF treatment led to distinct gene expression patterns in these two cell types. In particular, IGFBP3, a secreted protein previously shown to be, at least in some circumstances, an angiogenesis inhibitor (39) was upregulated by approximately 8 fold in BAE but not in BCE cells, a finding subsequently confirmed by qRT-PCR. However, recombinant IGFBP3 had no effects on BAE cell growth. Moreover, the conditioned medium of BAE cells treated with LIF for 72 hours did not inhibit BAE cells growth in the presence of a LIF neutralizing antibody, arguing against the hypothesis that LIF induced BAE growth inhibition was mediated by secreted factors. It was previously reported that STAT3 can induce caspase-independent cell death via upregulation of lysosomal proteases cathepsins B and L (40). Therefore, whether LIF might elicit such a signaling cascade in BAE cells was examined. Interestingly, CTSL but not CTSB was dramatically upregulated by LIF at both mRNA and protein levels by 24 hours of treatment in the BAE cells (FIGS. 6C and 6D). Co-incubation with CA074me, an inhibitor antagonizing both cathepsin B and L, alleviated LIF-induced growth inhibition in BAE cells in a dose-dependent manner of CA074me (FIG. 6E). Moreover, another cathepsin L-specific inhibitor, CAA0225, also repressed LIF-induced growth inhibition in BAE cells although by less extent (FIG. 6F). In contrast, the cathepsin B-selective inhibitor CA074 was not able to suppress LIF-induced effects in BAE cells even at the highest dose tested, 50 μM. Interestingly, cathepsin L mRNA (CTSL) levels in BCE cells were not detectable by qRT-PCR no matter when the cells were incubated with vehicle or LIF. These data collectively indicated that LIF induced upregulation of cathepsin L in BAE cells and in turn led to caspase-independent cell death. Moreover, after incubation with LIF for 48 hours, BAE cells showed significantly reduced BrdU incorporation compared to the vehicle control (FIGS. 7A and 7B), suggesting that cell cycle arrest was elicited by LIF. This notion was supported by downregulation of cyclin A/B in LIF-treated BAE but not in BCE cells (FIG. 7C).

In embodiments, this invention provides a novel and unexpected activity of IL-6 cytokines, such as LIF and CT-1, to induce blood vessel growth, i.e., angiogenesis.

For example, the invention provides that LIF, a molecule that has previously been characterized as an inhibitor of endothelial cell growth, has unexpected pro-angiogenic properties in the eye as assess by in vitro, ex vivo and in vivo studies.

The invention provides that LIF is able to directly stimulate the proliferation of choroidal endothelial cells, while it inhibits the growth of aortic endothelial cells, emphasizing the specificity and uniqueness of its effects on endothelial cells. LIF also promoted endothelial sprouting from choroidal explants and angiogenesis when injected into the mouse vitreous.

LIF is a well-characterized cytokine, member of the IL6 family. It interacts with the LIF receptor, which in turn forms heterodimers with GP130, resulting, among other effects, in Stat3 activation.

The invention provides that LIF can promote growth of a subset of endothelial cells offers opportunities for therapeutic intervention in a variety of conditions, including low perfusion in the retina/choroid, coronary artery and myocardial diseases (Reboucas et al., 2016; Simon-Yarza et al., 2012; Wang et al., 2013). The observation that LIF does not induce vascular permeability suggests that administration of this factor will avoid the undesirable vascular leakage associated with VEGF (Niu et al., 2016).

The invention suggests that IL-6 family members such as LIF and CT-1 can protect RPE from damage, including damage due to oxidative stress. This should represent a novel therapeutic strategy for treatment of retinal conditions associated with RPE damage or degeneration.

### REFERENCES

1. A. S. Chung, N. Ferrara, Developmental and pathological angiogenesis. Annual review of cell and developmental biology 27, 563-584 (2011).
2. M. Potente, H. Gerhardt, P. Carmeliet, Basic and therapeutic aspects of angiogenesis. Cell 146, 873-887 (2011).
3. G. D. Yancopoulos et al., Vascular-specific growth factors and blood vessel formation. Nature 407, 242-248 (2000).
4. N. Ferrara, A. P. Adamis, Ten years of anti-vascular endothelial growth factor therapy. Nat Rev Drug Discov 15, 385-403 (2016).
5. R. S. Apte, D. S. Chen, N. Ferrara, VEGF in Signaling and Disease: Beyond Discovery and Development. Cell 176, 1248-1264 (2019).
6. J. M. Isner, Vascular endothelial growth factor: gene therapy and therapeutic angiogenesis. Am J Cardiol 82, 63S-64S (1998).
7. M. Simons, Angiogenesis: where do we stand now? Circulation 111, 1556-1566 (2005).
8. S. Das, P. A. Marsden, Angiogenesis in glioblastoma. The New England journal of medicine 369, 1561-1563 (2013).
9. C. Depner et al., EphrinB2 repression through ZEB2 mediates tumour invasion and anti-angiogenic resistance. Nature communications 7, 12329 (2016).
10. R. Ferla, M. Bonomi, L. Otvos, Jr., E. Surmacz, Glioblastoma-derived leptin induces tube formation and growth of endothelial cells: comparison with VEGF effects. BMC cancer 11, 303 (2011).
11. A. Holm, T. Heumann, H. G. Augustin, Microvascular Mural Cell Organotypic Heterogeneity and Functional Plasticity. Trends Cell Biol 28, 302-316 (2018).
12. N. A. Nicola, J. J. Babon, Leukemia inhibitory factor (LIF). Cytokine Growth Factor Rev 26, 533-544 (2015).
13. G. X. Rosario, C. L. Stewart, The Multifaceted Actions of Leukaemia Inhibitory Factor in Mediating Uterine Receptivity and Embryo Implantation. Am J Reprod Immunol 75, 246-255 (2016).
14. J. Ash, D. S. McLeod, G. A. Lutty, Transgenic expression of leukemia inhibitory factor (LIF) blocks normal vascular development but not pathological neovascularization in the eye. Molecular vision 11, 298-308 (2005).
15. M. S. Pepper, N. Ferrara, L. Orci, R. Montesano, Leukemia inhibitory factor (LIF) inhibits angiogenesis in vitro. J Cell Sci 108 (Pt 1), 73-83 (1995).
16. Y. Kubota, M. Hirashima, K. Kishi, C. L. Stewart, T. Suda, Leukemia inhibitory factor regulates microvessel density by modulating oxygen-dependent VEGF expression in mice. The Journal of clinical investigation 118, 2393-2403 (2008).
17. Y. Takahashi et al., Leukemia inhibitory factor regulates trophoblast giant cell differentiation via Janus kinase 1-signal transducer and activator of transcription 3-suppressor of cytokine signaling 3 pathway. Mol Endocrinol 22, 1673-1681 (2008).
18. S. J. Rodig et al., Disruption of the Jak1 gene demonstrates obligatory and nonredundant roles of the Jaks in cytokine-induced biologic responses. Cell 93, 373-383 (1998).
19. S. G. Rane, E. P. Reddy, Janus kinases: components of multiple signaling pathways. Oncogene 19, 5662-5679 (2000).
20. H. Kiu, S. E. Nicholson, Biology and significance of the JAK/STAT signalling pathways. Growth Factors 30, 88-106 (2012).
21. H. Yu, D. Pardoll, R. Jove, STATs in cancer inflammation and immunity: a leading role for STAT3. Nature reviews. Cancer 9, 798-809 (2009).
22. J. R. McColm, P. Geisen, L. J. Peterson, M. E. Hartnett, Exogenous leukemia inhibitory factor (LIF) attenuates retinal vascularization reducing cell proliferation not apoptosis. Experimental eye research 83, 438-446 (2006).
23. Z. Shao et al., Choroid sprouting assay: an ex vivo model of microvascular angiogenesis. PloS one 8, e69552 (2013).
24. M. Atanasova, A. Whitty, Understanding cytokine and growth factor receptor activation mechanisms. Critical reviews in biochemistry and molecular biology 47, 502-530 (2012).
25. D. Pennica et al., Cardiotrophin-1. Biological activities and binding to the leukemia inhibitory factor receptor/gp130 signaling complex. The Journal of biological chemistry 270, 10915-10922 (1995).
26. D. Pennica et al., Expression cloning of cardiotrophin 1, a cytokine that induces cardiac myocyte hypertrophy. Proceedings of the National Academy of Sciences of the United States of America 92, 1142-1146 (1995).
27. D. P. Gearing, A. G. Bruce, Oncostatin M binds the high-affinity leukemia inhibitory factor receptor. New Biol 4, 61-65 (1992).
28. J. Hanus, C. Anderson, D. Sarraf, J. Ma, S. Wang, Retinal pigment epithelial cell necroptosis in response to sodium iodate. Cell Death Discov 2, 16054 (2016).
29. A. Mizota, E. Adachi-Usami, Functional recovery of retina after sodium iodate injection in mice. Vision Res 37, 1859-1865 (1997).
30. P. O. Magalhaes et al., Methods of endotoxin removal from biological preparations: a review. Journal of pharmacy & pharmaceutical sciences: a publication of the Canadian Society for Pharmaceutical Sciences, Societe canadienne des sciences pharmaceutiques 10, 388-404 (2007).
31. S. Sakimoto et al., A role for endothelial cells in promoting the maturation of astrocytes through the apelin/APJ system in mice. Development 139, 1327-1335 (2012).
32. H. West, W. D. Richardson, M. Fruttiger, Stabilization of the retinal vascular network by reciprocal feedback between blood vessels and astrocytes. Development 132, 1855-1862 (2005).
33. C. Tao, X. Zhang, Development of astrocytes in the vertebrate eye. Developmental dynamics: an official publication of the American Association of Anatomists 243, 1501-1510 (2014).
34. L. J. Duan, S. J. Pan, T. N. Sato, G. H. Fong, Retinal Angiogenesis Regulates Astrocytic Differentiation in Neonatal Mouse Retinas by Oxygen Dependent Mechanisms. Scientific reports 7, 17608 (2017).
35. N. Ferrara, J. Winer, W. J. Henzel, Pituitary follicular cells secrete an inhibitor of aortic endothelial cell growth: identification as leukemia inhibitory factor. Proceedings of the National Academy of Sciences of the United States of America 89, 698-702 (1992).
36. H. Kojima, T. Inoue, H. Kunimoto, K. Nakajima, IL-6-STAT3 signaling and premature senescence. Jak-Stat 2, e25763 (2013).
37. M. Sapochnik, M. Fuertes, E. Arzt, Programmed cell senescence: role of IL-6 in the pituitary. Journal of molecular endocrinology 58, R241-R253 (2017).
38. R. Salama, M. Sadaie, M. Hoare, M. Narita, Cellular senescence and its effector programs. Genes & development 28, 99-114 (2014).
39. B. Liu et al., Insulin-like growth factor-binding protein-3 inhibition of prostate cancer growth involves suppression of angiogenesis. Oncogene 26, 1811-1819 (2007).
40. P. A. Kreuzaler et al., Stat3 controls lysosomal-mediated cell death in vivo. Nature cell biology 13, 303-309 (2011).
41. S. Takashima, M. Klagsbrun, Inhibition of endothelial cell growth by macrophage-like U-937 cell-derived oncostatin M, leukemia inhibitory factor, and transforming growth factor beta1. The Journal of biological chemistry 271, 24901-24906 (1996).
42. M. Vasse et al., Oncostatin M induces angiogenesis in vitro and in vivo. Arterioscler Thromb Vasc Biol 19, 1835-1842 (1999).
43. T. Pannicke, L. Wagner, A. Reichenbach, A. Grosche, Electrophysiological characterization of Muller cells from the ischemic retina of mice deficient in the leukemia inhibitory factor. Neuroscience letters 670, 69-74 (2018).
44. D. M. Sherry, R. Mitchell, H. Li, D. R. Graham, J. D. Ash, Leukemia inhibitory factor inhibits neuronal development and disrupts synaptic organization in the mouse retina. J Neurosci Res 82, 316-332 (2005).
45. L. Bozoyan, J. Khlghatyan, A. Saghatelyan, Astrocytes control the development of the migration-promoting vasculature scaffold in the postnatal brain via VEGF signaling. The Journal of neuroscience: the official journal of the Society for Neuroscience 32, 1687-1704 (2012).
46. J. Chen, L. E. Smith, Retinopathy of prematurity. Angiogenesis 10, 133-140 (2007).
47. T. Alon et al., Vascular endothelial growth factor acts as a survival factor for newly formed retinal vessels and has implications for retinopathy of prematurity. Nature medicine 1, 1024-1028 (1995).
48. J. S. Penn et al., Vascular endothelial growth factor in eye disease. Progress in retinal and eye research 27, 331-371 (2008).
49. S. Pensa et al., Signal transducer and activator of transcription 3 and the phosphatidylinositol 3-kinase regulatory subunits p55α and p50α regulate autophagy in vivo. The FEBS journal 281, 4557-4567 (2014).
50. A. Kaasik, T. Rikk, A. Piirsoo, T. Zharkovsky, A. Zharkovsky, Up-regulation of lysosomal cathepsin L and autophagy during neuronal death induced by reduced serum and potassium. The European journal of neuroscience 22, 1023-1031 (2005).
51. J. Liu et al., Cathepsin L expression and regulation in human abdominal aortic aneurysm, atherosclerosis, and vascular cells. Atherosclerosis 184, 302-311 (2006).
52. W. Li, L. Kornmark, L. Jonasson, C. Forssell, X. M. Yuan, Cathepsin L is significantly associated with apoptosis and plaque destabilization in human atherosclerosis. Atherosclerosis 202, 92-102 (2009).
53. N. A. Gillett, D. Lowe, L. Lu, C. Chan, N. Ferrara, Leukemia inhibitory factor expression in human carotid plaques: possible mechanism for inhibition of large vessel endothelial regrowth. Growth Factors 9, 301-305 (1993).
54. M. Snyder, X. Y. Huang, J. J. Zhang, Identification of novel direct Stat3 target genes for control of growth and differentiation. The Journal of biological chemistry 283, 3791-3798 (2008).
55. M. Zhou, H. Yang, R. M. Learned, H. Tian, L. Ling, Non-cell-autonomous activation of IL-6/STAT3 signaling mediates FGF19-driven hepatocarcinogenesis. Nature communications 8, 15433 (2017).
56. R. L. Robinson et al., Comparative STAT3-Regulated Gene Expression Profile in Renal Cell Carcinoma Subtypes. Frontiers in oncology 9, 72 (2019).
57. C. Gong et al., Abnormally expressed JunB transactivated by IL-6/STAT3 signaling promotes uveal melanoma aggressiveness via epithelial-mesenchymal transition. Bioscience reports 38 (2018).
58. B. Jin et al., PIM-1 modulates cellular senescence and links IL-6 signaling to heterochromatin formation. Aging cell 13, 879-889 (2014).
59. J. LeCouter et al., Angiogenesis-independent endothelial protection of liver: role of VEGFR-1. Science 299, 890-893 (2003).
60. H. G. Augustin, G. Y. Koh, Organotypic vasculature: From descriptive heterogeneity to functional pathophysiology. Science 357 (2017).
61. D. J. Nolan et al., Molecular signatures of tissue-specific microvascular endothelial cell heterogeneity in organ maintenance and regeneration. Developmental cell 26, 204-219 (2013).
62. J. Kim et al., Tie2 activation promotes choriocapillary regeneration for alleviating neovascular age-related macular degeneration. Science advances 5, eaau6732 (2019).
63. L. Yu et al., Interaction between Bevacizumab and Murine VEGF-A: A Reassessment. Invest Ophthalmol Vis Sci 49, 522-527 (2008).
64. H. Xin, C. Zhong, E. Nudleman, N. Ferrara, Evidence for Pro-angiogenic Functions of VEGF-Ax. Cell 167, 275-284 e276 (2016).
65. L. Scheppke et al., Retinal vascular permeability suppression by topical application of a novel VEGFR2/Src kinase inhibitor in mice and rabbits. The Journal of clinical investigation 118, 2337-2346 (2008).
66. J. Xu et al., Evaluating changes of blood flow in retina, choroid, and outer choroid in rats in response to elevated intraocular pressure by 1300 nm swept-source OCT. Microvascular research 121, 37-45 (2019).
67. Yue, X., Wu, L., and Hu, W. (2015). The regulation of leukemia inhibitory factor. Cancer cell & microenvironment 2.

What is claimed is:

1. A method of treatment for a condition related to inadequate vascularization in the eye of a subject comprising administering to a subject in need thereof an effective amount of an IL-6 family protein, or a functional fragment thereof, to promote angiogenesis.

2. The method of claim 1, wherein the administration increases retinal microvessel density.

3. The method of claim 1, wherein the administration increases proliferation of choroidal endothelial cells.

4. The method of claim 1, wherein the condition is age-related macular degeneration.

5. The method of claim 1, wherein the condition is retinopathy of prematurity (ROP).

6. The method of claim 1, wherein the administration is via intravitreal injection.

7. The method of claim 1, wherein the effective amount does not induce vascular leakage.

8. The method of claim 1, wherein the effective amount does not induce edema.

9. The method of claim 1, wherein the IL-6 family protein is leukemia inhibitory factor (LIF).

10. The method of claim 1, wherein the IL-6 family protein is cardiotrophin-1 (CT-1).

11. A method of inducing blood vessel formation in the eye of a subject comprising administering to a subject in need thereof an effective amount of an IL-6 family protein, or a functional fragment thereof.

12. The method of claim 11, wherein the administration increases retinal angiogenesis.

13. The method of claim 10, wherein the administration increases proliferation of choroidal endothelial cells.

14. The method of claim 10, wherein the subject has age-related macular degeneration.

15. The method of claim 10, wherein the subject has retinopathy of prematurity (ROP).

16. The method of claim 10, wherein the administration is via intravitreal injection.

17. The method of claim 10, wherein the effective amount does not induce vascular leakage.

18. The method of claim 10, wherein the effective amount does not induce edema.

19. The method of claim 10, wherein the IL-6 family protein is leukemia inhibitory factor (LIF).

20. The method of claim 10, wherein the IL-6 family protein is cardiotrophin-1 (CT-1).
